(19) United States
(12) Patent Application Publication
(10) Pub. No.: US 2025/0134601 A1
(43) Pub. Date: May 1, 2025

(54) METHODS AND SYSTEMS FOR ALIGNING A PROSTHETIC VALVE WITH A NATIVE VALVE

(71) Applicant: EDWARDS LIFESCIENCES CORPORATION, Irvine, CA (US)

(72) Inventor: John Webb, Vancouver (CA)

(73) Assignee: EDWARDS LIFESCIENCES CORPORATION, Irvine, CA (US)

(21) Appl. No.: 19/007,832

(22) Filed: Jan. 2, 2025

Related U.S. Application Data
(63) Continuation of application No. PCT/US2023/027179, filed on Jul. 8, 2023.
(60) Provisional application No. 63/388,210, filed on Jul. 11, 2022.

(51) Int. Cl. A61B 34/20; A61B 90/00; A61F 2/24; A61M 25/01

(52) U.S. Cl. CPC A61B 34/20; A61F 2/2418; A61F 2/2433; A61B 2090/376; A61B 2090/3966; A61M 25/0108; A61M 2025/0166

(57) ABSTRACT

Methods and systems for rotationally aligning cusps of a prosthetic heart valve with cusps of a native valve are disclosed. As one example, a method includes advancing a distal end portion of a delivery apparatus toward a native valve of a heart, a prosthetic heart valve radially compressed around the distal end portion, and visualizing under fluoroscopy, within an imaging view, a position one or more radiopaque markers disposed on the distal end portion relative to one or more native valve cusps. The method further includes rotating the distal end portion until the one or more markers are circumferentially aligned with a middle of the one or more native valve cusps and deploying the radially compressed prosthetic heart valve with the delivery apparatus to radially expand the prosthetic heart valve in the native valve such that the cusps of the prosthetic heart valve and the native valve are circumferentially aligned.

### CROSS-REFERENCE TO RELATED APPLICATIONS

This application is a continuation of PCT Application No. PCT/US2023/027179, filed Jul. 8, 2023, which claims the benefit of U.S. Provisional Patent Application No. 63/388,210, filed Jul. 11, 2022, the entire contents of each of which are incorporated by reference herein.

### FIELD

The present disclosure relates to prosthetic heart valves and methods for deploying a radially expandable prosthetic heart valve at a native valve with a delivery apparatus such that leaflet cusps and commissure of the radially expanded prosthetic heart valve are aligned with leaflet cusps and commissures of the native valve.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery device and advanced through the patient's vasculature (for example, through a femoral artery and the aorta) until the prosthetic valve reaches the implantation site in the heart. The prosthetic valve is then expanded to its functional size, for example, by inflating a balloon on which the prosthetic valve is mounted.

When deploying the prosthetic valve at the native valve by inflating the balloon of the delivery device (or by another deployment mechanism of the delivery apparatus), the radially expanded prosthetic valve is deployed at a random radial orientation relative to the native valve. In some examples, the positioning of an implanted prosthetic heart valve relative to the native anatomy can affect the performance of the prosthetic heart valve, the function of the native anatomy, or the ability to perform future interventions.

Accordingly, a need exists for improved delivery apparatuses and methods for positioning radially expandable prosthetic heart valves relative to the native anatomy.

### SUMMARY

Described herein are examples of systems and methods for delivering a prosthetic valve to and implanting the prosthetic valve at a native valve of a heart of a patient with leaflet cusps of the prosthetic valve in alignment with leaflet cusps or with commissures of the prosthetic valve in alignment with commissures of the native valve. In some examples, the prosthetic valve can be mounted in a radially compressed state onto a delivery apparatus for delivery to a target implantation site and then deployed at the target implantation site, in the native valve, with the delivery apparatus. In some examples, the delivery apparatus can include an inflatable balloon and the prosthetic valve can be radially expanded and deployed by inflating the balloon at the target implantation site. After reaching the native valve, one or more markers configured to be visualized under fluoroscopic imaging, which are disposed on a distal end portion of the delivery apparatus and correspond to a location of one or more cusps of the prosthetic valve, can be aligned with one or more cusps of the native valve in the imaging view such that after deploying the prosthetic valve, the cusps of the prosthetic valve are aligned (for example, in a circumferential direction) with cusps of the native valve.

A method can comprise advancing a distal end portion of a delivery apparatus toward a native valve of a heart, where a prosthetic heart valve is radially compressed around a valve mounting portion of the distal end portion of the delivery apparatus.

In some examples, the method can comprise visualizing under long axis fluoroscopy, within an imaging view, a position one or more radiopaque markers relative to one or more cusps of the native valve, the one or more radiopaque markers disposed on the distal end portion of the delivery apparatus, and where at least one radiopaque marker of the one or more radiopaque markers corresponds to a location of a specified cusp of the prosthetic heart valve.

In some examples, the method can comprise rotating the distal end portion of the delivery apparatus until the one or more markers are circumferentially aligned with a middle of the one or more cusps of the native valve, and deploying the radially compressed prosthetic heart valve with the delivery apparatus to radially expand and implant the prosthetic heart valve in an annulus of the native valve such that the cusps of the prosthetic heart valve are circumferentially aligned with the cusps of the native valve.

In some examples, the method can comprise visualizing under long axis fluoroscopy, with a cusp overlap imaging view, a position of a radiopaque marker relative to a non-coronary cusp of the native valve that is disposed on a left side of the imaging view while a right coronary cusp and left coronary cusp of the native valve are superimposed with one another on a right side of the imaging view.

In some examples, the method can comprise rotating the distal end portion of the delivery apparatus until the radiopaque marker appears on a far-left side of the imaging view at a middle of the non-coronary cusp, and deploying the radially compressed prosthetic heart valve with the delivery apparatus to radially expand and implant the prosthetic heart valve in an annulus of the native valve.

In some examples, the method can comprise obtaining a 3D image of a heart and based on the obtained 3D image, selecting a long axis fluoroscopic imaging view that positions a right coronary cusp of a native valve of the heart in a center of the imaging view, anterior to a left-non commissure of the native valve.

In some examples, the method can comprise visualizing under long axis fluoroscopy, within the selected imaging view, a position of a radiopaque marker relative to the right coronary cusp of the native valve, the radiopaque marker disposed on the distal end portion of the delivery apparatus and corresponding to a location of a specified cusp of the prosthetic heart valve, rotating the distal end portion of the delivery apparatus until the radiopaque marker appears to be positioned anteriorly in the imaging view and is aligned with the middle of the right coronary cusp, and deploying the radially compressed prosthetic heart valve with the delivery apparatus to radially expand and implant the prosthetic heart valve in an annulus of the native valve.

In some examples, a method comprises advancing a distal end portion of a delivery apparatus toward a native valve of a heart, where a prosthetic heart valve is radially compressed around a valve mounting portion of the distal end portion of the delivery apparatus. The method further includes visualizing under long axis fluoroscopy, within an imaging view, a position one or more radiopaque markers relative to one or more cusps of the native valve, the one or more radiopaque markers disposed on the distal end portion of the delivery apparatus, and where at least one radiopaque marker of the one or more radiopaque markers corresponds to a location of a specified cusp of the prosthetic heart valve. The method further includes at or proximate to the native valve, rotating the distal end portion of the delivery apparatus until the one or more markers are circumferentially aligned with a middle of the one or more cusps of the native valve, and deploying the radially compressed prosthetic heart valve with the delivery apparatus to radially expand and implant the prosthetic heart valve in an annulus of the native valve such that the cusps of the prosthetic heart valve are circumferentially aligned with the cusps of the native valve.

In some examples, a method comprises advancing a distal end portion of a delivery apparatus toward a native valve of a heart, where a prosthetic heart valve is radially compressed around a valve mounting portion of the distal end portion of the delivery apparatus. The method further includes visualizing under long axis fluoroscopy, with a cusp overlap imaging view, a position of a radiopaque marker relative to a non-coronary cusp of the native valve that is disposed on a left side of the imaging view while a right coronary cusp and left coronary cusp of the native valve are superimposed with one another on a right side of the imaging view, the radiopaque marker disposed on the distal end portion of the delivery apparatus and corresponding to a location of a specified cusp of the prosthetic heart valve. The method further includes at or proximate to the native valve, rotating the distal end portion of the delivery apparatus until the radiopaque marker appears on a far left side of the imaging view at a middle of the non-coronary cusp, and deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

In some examples, a method comprises obtaining a 3D image of a heart and based on the obtained 3D image, selecting a long axis fluoroscopic imaging view that positions a right coronary cusp of a native valve of the heart in a center of the imaging view, anterior to a left-non commissure of the native valve. The method further includes advancing a distal end portion of a delivery apparatus toward the native valve of the heart, where a prosthetic heart valve is radially compressed around a valve mounting portion of the distal end portion of the delivery apparatus, and visualizing under long axis fluoroscopy, within the selected imaging view, a position of a radiopaque marker relative to the right coronary cusp of the native valve, the radiopaque marker disposed on the distal end portion of the delivery apparatus and corresponding to a location of a specified cusp of the prosthetic heart valve. The method further includes at or proximate to the native valve, rotating the distal end portion of the delivery apparatus until the radiopaque marker appears to be positioned anteriorly in the imaging view and is aligned with the middle of the right coronary cusp, and deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

In some examples, a method comprises one or more of the features recited in Examples 1-40 below.

The above method(s) can be performed on a living animal or on a simulation, such as on a cadaver, cadaver heart, anthropomorphic ghost, or simulator (e.g., with body parts, heart, tissue, etc. being simulated).

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2A is a perspective view of a prosthetic heart valve, according to another example.

FIG. 2B is a perspective view of the prosthetic valve of FIG. 2A with the components on the outside of the frame shown in transparent lines for purpose of illustration.

FIG. 3 is a side view of a delivery apparatus for implanting a prosthetic heart valve, according to one example.

FIG. 4 is a side view of a section of the handle and a section of the distal end portion of the delivery apparatus of FIG. 3.

FIG. 5 is a side view of the distal end portion of the delivery apparatus of FIG. 3.

FIG. 6 is a side view of the distal end portion of the delivery apparatus of FIG. 3 showing the inflatable balloon in an inflated state.

FIG. 7 is an enlarged cross-sectional view of the distal end portion of the delivery apparatus of FIG. 3 showing a prosthetic heart valve radially compressed around the inflatable balloon.

FIG. 8 is a schematic of an exemplary heart showing a position of coronary arteries relative to an aortic valve.

FIG. 9A illustrates an exemplary positioning of a prosthetic valve in an aortic valve, relative to a coronary artery.

FIG. 9B illustrates another exemplary positioning of a prosthetic valve in an aortic valve, relative to a coronary artery, where the prosthetic valve at least partially inhibits blood flow or catheter access to the coronary artery.

FIG. 10A is a cross-sectional view of an aortic valve illustrating a first positioning of a prosthetic valve within the aortic valve where commissures of the prosthetic valve at least partially block one or more openings to the coronary arteries.

FIG. 10B is a cross-sectional view of an aortic valve illustrating a second positioning of a prosthetic valve within the aortic valve where commissures of the prosthetic valve are circumferentially aligned with native commissure of the aortic valve, thereby maintaining access to the coronary arteries.

FIG. 11 illustrates a leaflet-cutting procedure where a leaflet of a native aortic valve can be split at a location of an entrance to a coronary artery when a prosthetic heart valve is implanted within the aortic valve to enable increased blood flow to enter the coronary artery.

FIG. 12A illustrates an exemplary transcatheter prosthetic heart valve and an example of how splitting surgical prosthetic leaflets surrounding the transcatheter prosthetic heart valve at a region of a frame of the surgical prosthetic heart valve that is between two adjacent commissures results in open cells in front of an entrance to a coronary artery.

FIG. 12B illustrates the exemplary transcatheter prosthetic heart valve of FIG. 12A and how splitting the surgical prosthetic leaflet overlying a coronary ostium does not result in normal coronary access or blood flow if the commissure of the transcatheter valve is a positioned adjacent to the split in the surgical leaflet (for example, the cusps or commissures of the two valves are not aligned).

FIG. 13 is an exemplary fluoroscopic image of a native aortic valve viewed with long axis fluoroscopy in a standard, three-cusp imaging view.

FIG. 14 is a schematic of an exemplary view of a native aortic valve that can be obtained with short axis CT imaging.

FIG. 15 is a schematic of an exemplary fluoroscopic image obtained during a prosthetic heart valve implantation procedure showing an axial position of a distal end portion of a delivery apparatus relative to the native valve.

FIG. 16 is a schematic of an exemplary CT image showing a radially compressed prosthetic heart valve on a delivery apparatus within an annulus of the native valve.

FIG. 17 is a schematic of an exemplary fluoroscopic image showing a distal end portion of a delivery apparatus arranged at a native heart valve and two overlapping radiopaque markers on the distal end portion of the delivery apparatus.

FIG. 18 is a schematic of an exemplary CT image showing the distal end portion of the delivery apparatus arranged at the native heart valve and the two radiopaque markers on the distal end portion of the delivery apparatus which are disposed 180 degrees apart from one another around a circumference of the delivery apparatus.

FIGS. 19A-19C are sides views illustrating different rotational orientations of a shaft of a delivery apparatus, the shaft including a pair of radiopaque markers that can be mounted on or embedded within a shaft, 180 degrees apart from one another around a circumference of the shaft, according to an example.

FIGS. 20A-20C are side views illustrating different rotational orientations of a shaft of a delivery apparatus, the shaft including a pair of radiopaque markers that can be mounted on or embedded within a shaft, 180 degrees apart from one another around a circumference of the shaft, according to another example.

FIG. 21 is a schematic of an exemplary fluoroscopic image showing a distal end portion of a delivery apparatus arranged at a native heart valve and a single asymmetric radiopaque marker on the distal end portion of the delivery apparatus.

FIG. 22 is a schematic showing the distal end portion of the delivery apparatus arranged at the native heart valve and the single asymmetric marker on the distal end portion of the delivery apparatus.

FIG. 23 is a schematic showing the distal end portion of the delivery apparatus arranged at the native heart valve and three radiopaque markers on the distal end portion of the delivery apparatus which are disposed 120 degrees apart from one another around a circumference of the delivery apparatus.

FIG. 24 is a schematic showing a prosthetic heart valve radially expanded within a native heart valve with cusps of the prosthetic heart valve aligned with cusps of the native heart valve.

FIG. 25 is a schematic of an exemplary image showing a 3D arrangement of cusps of a native heart valve in a cusp overlap fluoroscopic imaging view.

FIG. 26 is a schematic of an exemplary fluoroscopic image of the cusp overlap imaging view of a native heart valve.

FIG. 27 is a flow chart of a method for implanting a prosthetic heart valve with cusps circumferentially aligned with the native cusps of a native heart valve.

### DETAILED DESCRIPTION

### General Considerations

For purposes of this description, certain aspects, advantages, and novel features of the examples of this disclosure are described herein. The described methods, systems, and apparatus should not be construed as limiting in any way. Instead, the present disclosure is directed toward all novel and non-obvious features and aspects of the various disclosed examples, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present, or problems be solved.

Features, integers, characteristics, compounds, chemical moieties, or groups described in conjunction with a particular aspect or example of the disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract, and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

As used herein, the terms “a,” “an,” and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A,” “B,” “C,” “A and B,” “A and C,” “B and C,” or “A, B, and C.”

As used herein, the term “coupled” generally means physically coupled or linked and does not exclude the presence of intermediate elements between the coupled items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, with reference to the prosthetic heart valve and the delivery apparatus, “proximal” refers to a position, direction, or portion of a component that is closer to the user and/or a handle of the delivery apparatus that is outside the patient, while “distal” refers to a position, direction, or portion of a component that is further away from the user and/or the handle of the delivery apparatus and closer to the implantation site. The terms “longitudinal” and “axial” refer to an axis extending in the proximal and distal directions, unless otherwise expressly defined. Further, the term “radial” refers to a direction that is arranged perpendicular to the axis and points along a radius from a center of an object (where the axis is positioned at the center, such as the longitudinal axis of the prosthetic valve).

As used herein, “e.g.” means “for example,” and “i.e.” means “that is.”

### Examples of the Disclosed Technology

Described herein are examples of prosthetic valve delivery apparatuses and methods for delivering and implanting a radially expandable prosthetic valve at a native valve of a heart such that leaflet cusp of the prosthetic valve are circumferentially aligned with leaflet cusps of the native valve, thereby resulting in commissure alignment between the prosthetic valve and native valve.

In some examples, a delivery apparatus can include a handle portion, a balloon catheter extending distally from the handle portion, and an inflatable balloon mounted at a distal end portion of the balloon catheter. In some examples, the balloon catheter can include a valve mounting portion configured to receive a radially compressed prosthetic heart valve, the valve mounting portion including or disposed adjacent to a portion of the inflatable balloon. In some examples, the balloon catheter can be configured to rotate relative to the handle portion. Further, in some examples, the delivery apparatus can include one or more radiopaque markers mounted on or embedded within a distal end portion of a shaft of the balloon catheter and which correspond to a location of one or more leaflet cusps of the prosthetic heart valve.

In this way, the delivery apparatus can be configured to allow a user to visualize under standard fluoroscopy, during an implantation procedure, the one or more radiopaque markers relative to the native anatomy. For example, a user may rotate the distal end portion of the delivery apparatus including the radially compressed prosthetic heart valve until the one or more radiopaque markers appear anteriorly and appear to overlap a middle of one or more cusps of the native valve in the imaging view. As a result, the prosthetic heart valve can be implanted in the annulus of the native valve with leaflet cusps in alignment with the native valve, thereby enabling future interventional procedures that require access to coronary arteries or the valve leaflets.

Prosthetic valves disclosed herein can be radially compressible and expandable between a radially compressed configuration and a radially expanded configuration. Thus, the prosthetic valves can be crimped on a delivery apparatus in the radially compressed configuration during delivery, and then expanded to the radially expanded configuration once the prosthetic valve reaches the implantation site. In some examples, the prosthetic valve can be deployed from the delivery apparatus at the implantation site (for example, a native valve of a heart) via inflating an inflatable balloon of the delivery apparatus.

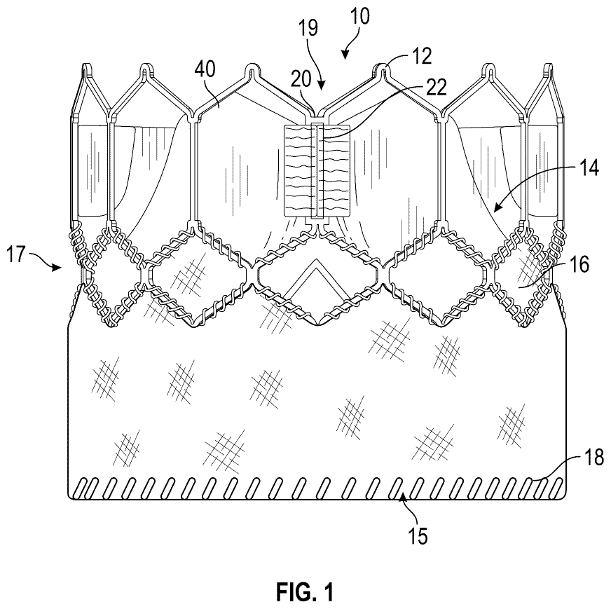

FIG. 1 shows a prosthetic heart valve (prosthetic valve) 10, according to one example. The illustrated prosthetic valve is adapted to be implanted in the native aortic annulus, although in other examples it can be adapted to be implanted in the other native annuluses of the heart (for example, the pulmonary, mitral, and tricuspid valves). The prosthetic valve can also be adapted to be implanted in other tubular organs or passageways in the body. The prosthetic valve 10 can have four main components: a stent or frame 12, a valvular structure 14, an inner skirt 16, and a perivalvular outer sealing member or outer skirt 18. The prosthetic valve 10 can have an inflow end portion 15, an intermediate portion 17, and an outflow end portion 19.

The valvular structure 14 can comprise three leaflets 40, collectively forming a leaflet structure, which can be arranged to collapse in a tricuspid arrangement, although in other examples there can be greater or fewer number of leaflets. The leaflets 40 can be secured to one another at their adjacent sides to form commissures 22 of the valvular structure 14. The lower edge of valvular structure 14 can have an undulating, curved scalloped shape and can be secured to the inner skirt 16 by sutures (not shown). In some examples, the leaflets 40 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein.

The frame 12 can be formed with a plurality of circumferentially spaced slots, or commissure windows 20 that are adapted to mount the commissures 22 of the valvular structure 14 to the frame. The frame 12 can be made of any of various suitable plastically-expandable materials (for example, stainless steel, etc.) or self-expanding materials (for example, Nitinol), as known in the art. When constructed of a plastically-expandable material, the frame 12 (and thus the prosthetic valve 10) can be crimped to a radially collapsed configuration on a delivery catheter and then expanded inside a patient by an inflatable balloon or equivalent expansion mechanism. When constructed of a self-expandable material, the frame 12 (and thus the prosthetic valve 10) can be crimped to a radially collapsed configuration and restrained in the collapsed configuration by insertion into a sheath or equivalent mechanism of a delivery catheter. Once inside the body, the prosthetic valve can be advanced from the delivery sheath, which allows the prosthetic valve to expand to its functional size.

Suitable plastically-expandable materials that can be used to form the frame 12 include, metal alloys, polymers, or combinations thereof. Example metal alloys can comprise one or more of the following: nickel, cobalt, chromium, molybdenum, titanium, or other biocompatible metal. In some examples, the frame 12 can comprise stainless steel. In some examples, the frame 12 can comprise cobalt-chromium. In some examples, the frame 12 can comprise nickel-cobalt-chromium. In some examples, the frame 12 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

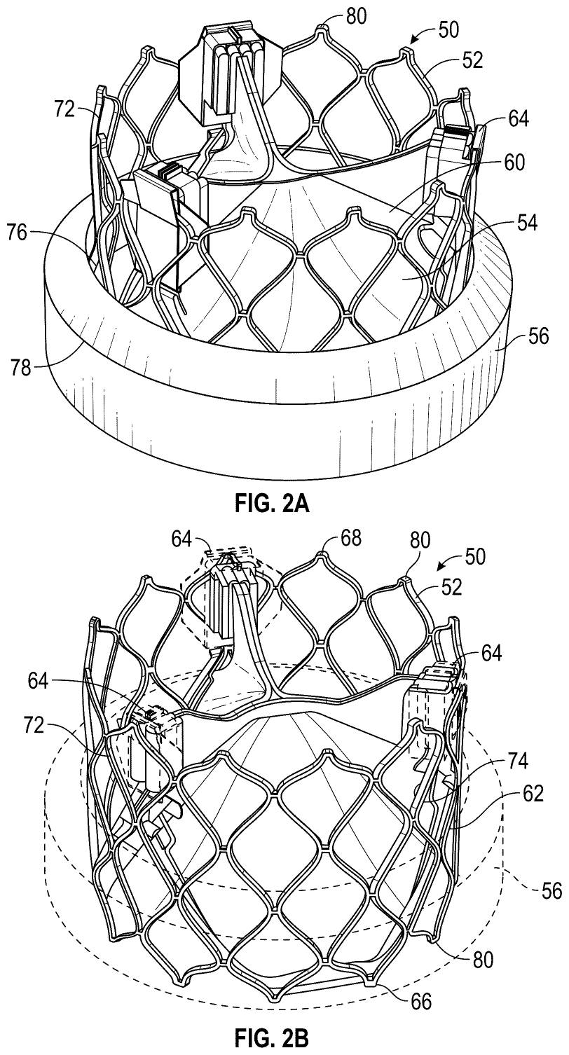

FIG. 2A is a perspective view of a prosthetic heart valve 50, according to another example. The prosthetic valve 50 can have three main components: a stent or frame, 52, a valvular structure 54, and a sealing member 56. FIG. 2B is a perspective view of the prosthetic valve 50 with the components on the outside of the frame 52 (including the sealing member 56) shown in transparent lines for purposes of illustration.

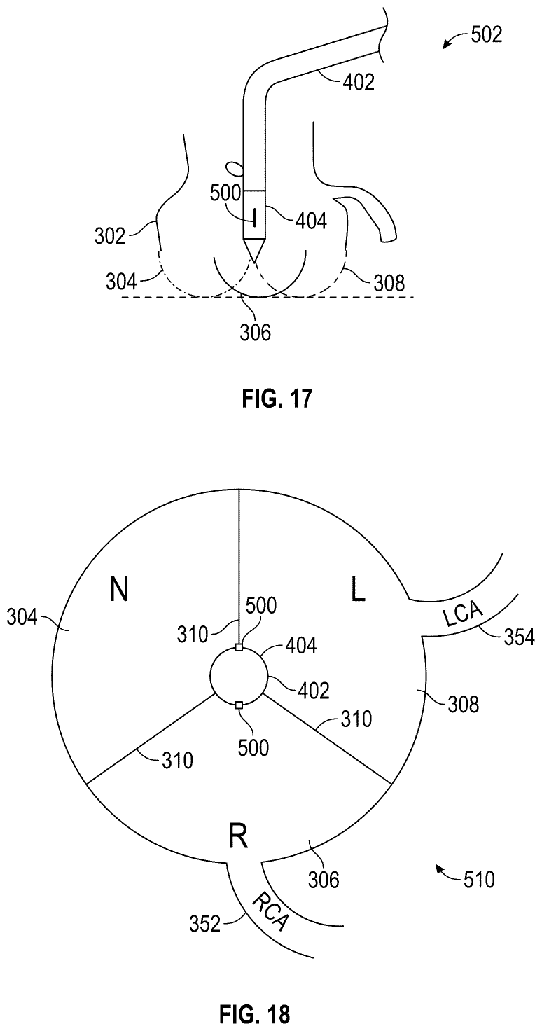

Like the valvular structure 14 of FIG. 1, the valvular structure 54 can comprise three leaflets 60, collectively forming a leaflet structure, which can be arranged to collapse in a tricuspid arrangement. Each leaflet 60 can be coupled to the frame 52 along its inflow edge 62 (the lower edge in the figures; also referred to as “cusp edges”) and at commissures 64 of the valvular structure 54 where adjacent portions (for example, commissure tabs) of two leaflets are connected to each other. In some examples, the commissures 64 can comprise an attachment member (for example, comprising fabric, flexible polymer, or the like) arranged across a cell (commissure cell) of the frame 52, the cell formed by struts of the frame. The attachment member can be secured to the struts of the frame forming the cell and the adjacent portions of the two leaflets can be connected to the attachment member to form the commissure 64 (for example, as shown in FIGS. 16 and 17, as described further below).

A reinforcing element (not shown), such as a fabric strip, can be connected directly to the cusp edges of the leaflets and to the struts of the frame to couple the cusp edges of the leaflets to the frame.

Similar to the frame 12 of FIG. 1, the frame 52 can be made of any of various suitable plastically-expandable materials or self-expanding materials, as known in the art and described above. The frame 52 in the illustrated example comprises a plurality of circumferentially extending rows of angled struts 72 defining rows of cells, or openings, 74 of the frame. The frame 52 can have a cylindrical or substantially cylindrical shape having a constant diameter from an inflow end 66 to an outflow end 68 of the frame as shown, or the frame can vary in diameter along the height of the frame, as disclosed in U.S. Patent Publication No. 2012/0239142, which is incorporated herein by reference.

The frame 52, at each of the inflow end 66 and the outflow end 68, may comprise a plurality of apices 80 spaced apart from one another around a circumference of the frame 52.

The sealing member 56 in the illustrated example is mounted on the outside of the frame 52 and functions to create a seal against the surrounding tissue (for example, the native leaflets and/or native annulus) to prevent or at least minimize paravalvular leakage. The sealing member 56 can comprise an inner layer 76 (which can be in contact with the outer surface of the frame 52) and an outer layer 78. The sealing member 56 can be connected to the frame 52 using suitable techniques or mechanisms. For example, the sealing member 56 can be sutured to the frame 52 via sutures that can extend around the struts 72 and through the inner layer 76. In alternative examples, the inner layer 76 can be mounted on the inner surface of the frame 52, while the outer layer 78 is on the outside of the frame 52.

The outer layer 78 can be configured or shaped to extend radially outward from the inner layer 76 and the frame 52 when the prosthetic valve 50 is deployed. When the prosthetic valve is fully expanded outside of a patient's body, the outer layer 78 can expand away from the inner layer 76 to create a space between the two layers. Thus, when implanted inside the body, this allows the outer layer 78 to expand into contact with the surrounding tissue.

Additional details regarding the prosthetic valve 50 and its various components are described in U.S. Patent Publication No. 2018/0028310, which is incorporated herein by reference.

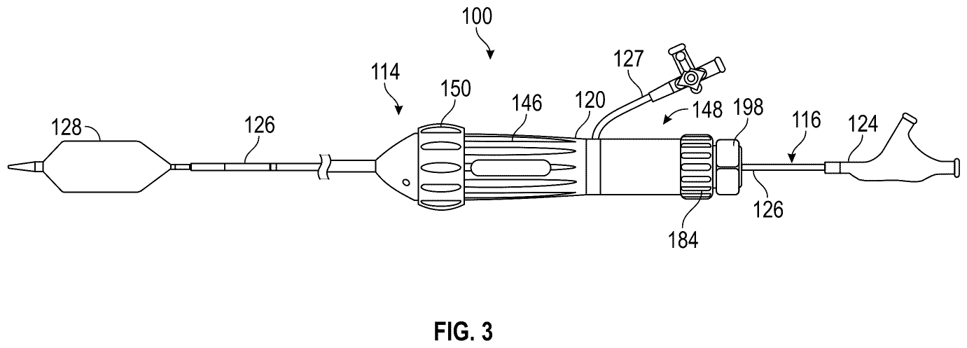

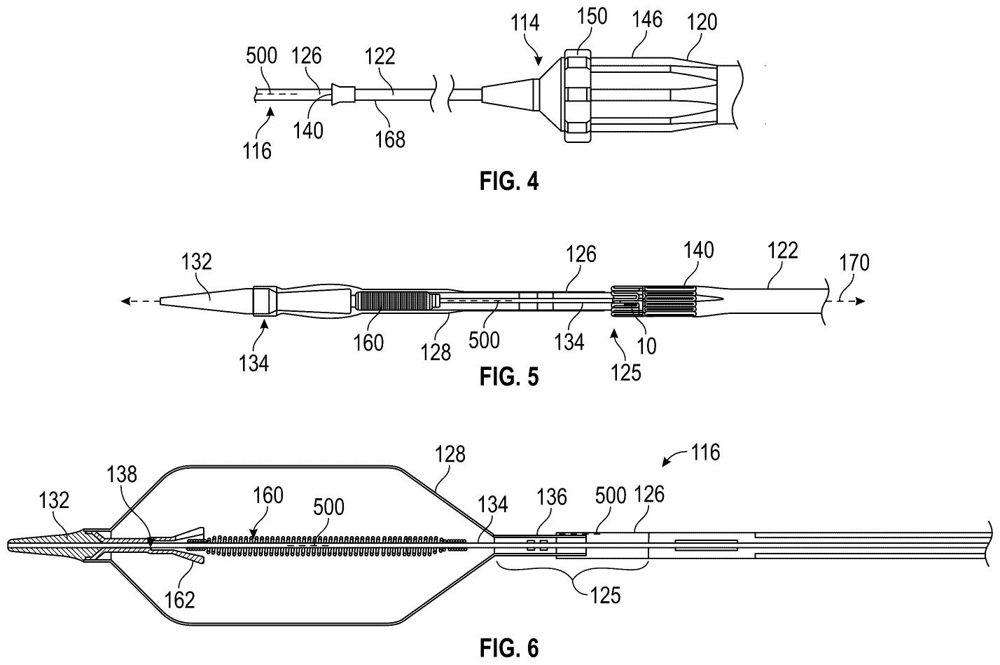

FIGS. 3-7 show a delivery apparatus 100 adapted to deliver and implant a prosthetic heart valve (for example, prosthetic valve 10 of FIG. 1 or 50 of FIGS. 2A-2B, with valve 10 shown schematically in FIG. 7 as an example) to a heart, according to one example. The delivery apparatus 100 generally includes a steerable guide catheter 114 (FIGS. 3 and 4), and a balloon catheter 116 extending through the guide catheter 114 (FIG. 3). The guide catheter 114 can also be referred to as a flex catheter or a main catheter. The use of the term main catheter should be understood, however, to include flex or guide catheters, as well as other catheters that do not have the ability to flex or guide through a patient's vasculature.

The guide catheter 114 and the balloon catheter 116 in the illustrated example are adapted to slide longitudinally relative to each other to facilitate delivery and positioning of the prosthetic valve 10 (or another prosthetic valve or expandable prosthetic medical device) at an implantation site in a patient's body, as described further below.

The guide catheter 114 includes a handle portion 120 (handle) and an elongated guide tube, or shaft, 122 extending from handle portion 120 (FIG. 4). FIG. 3 shows the delivery apparatus 100 without the guide catheter shaft 122 for purposes of illustration. FIG. 4 shows the guide catheter shaft 122 extending from the handle portion 120 over the balloon catheter 116. The balloon catheter 116 includes a proximal portion 124 (FIG. 3) adjacent the handle portion 120 and an elongated shaft 126 (or outer balloon catheter shaft) that extends from the proximal portion 124 and through handle portion 120 and guide catheter shaft 122. The handle portion 120 can include a side arm 127 (FIG. 3) having an internal passage which fluidly communicates with a lumen defined by the handle portion 120.

An inflatable balloon 128 is mounted at the distal end portion of balloon catheter 116 (FIGS. 5-7). In some examples, as shown in FIG. 5, the delivery apparatus 100 is configured to mount the prosthetic valve 10 in a crimped state proximal to the balloon 128 for insertion of the delivery apparatus 100 and prosthetic valve 10 into a patient's vasculature, which is described in detail in U.S. Pat. No. 9,061,119, which is incorporated herein by reference. Because prosthetic valve 10 can be crimped at a location different from the location of balloon 128 (for example, in this case prosthetic valve 10 is crimped proximal to balloon 128), prosthetic valve 10 can be crimped to a lower profile than would be possible if prosthetic valve 10 was crimped on top of balloon 128. This lower profile permits a surgeon to more easily navigate the delivery apparatus 100 (including crimped valve 10) through a patient's vasculature to the treatment location. The lower profile of the crimped prosthetic valve can be particularly helpful when navigating through portions of the patient's vasculature which are particularly narrow, such as the iliac artery. The lower profile also allows for treatment of a wider population of patients, with enhanced safety.

In alternate examples, the delivery apparatus 100 can be configured to mount the prosthetic valve directly on and around the balloon 128 (for example, in the position shown in FIG. 7).

A nose cone 132 (FIG. 5) can be mounted at the distal end of the delivery apparatus 100 to facilitate advancement of the delivery apparatus 100 through the patient's vasculature to the implantation site. In some instances, it may be useful to have nose cone 132 connected to a separate elongated shaft so that nose cone 132 can move independently of other elements of delivery apparatus 100. Nose cone 132 can be formed of a variety of materials, including various plastic materials.

As can be seen in FIGS. 5 and 6, the balloon catheter 116 in the illustrated configuration further includes an inner shaft 134 that extends from the proximal portion 124 and coaxially through the outer balloon catheter shaft 126 and the balloon 128, relative to a central longitudinal axis 170 of the delivery apparatus 100. The balloon 128 can be supported on a distal end portion of the inner shaft 134 that extends outwardly from the outer balloon catheter shaft 126 with a proximal end portion 136 of the balloon 128 secured to the distal end of the outer balloon catheter shaft 126 (for example, with a suitable adhesive) (FIG. 6). The outer diameter of the inner shaft 134 can be sized such that an annular space is defined between the inner shaft 134 and outer balloon catheter shaft 126 along the entire length of the outer balloon catheter shaft 126. The proximal portion 124 of the balloon catheter 116 can be formed with a fluid passageway that is fluidly connectable to a fluid source (for example, saline) for inflating the balloon 128. The fluid passageway is in fluid communication with the annular space between the inner shaft 134 and outer balloon catheter shaft 126 such that fluid from the fluid source can flow through the fluid passageway, through the space between the shafts, and into the balloon 128 to inflate the same and deploy prosthetic valve 10.

The proximal portion 124 can also define an inner lumen that is in communication with a lumen 138 of the inner shaft 134 (FIG. 6) that is sized to receive a guide wire that can extend coaxially through the inner shaft 134 and the nose cone 132.

The inner shaft 134 and outer balloon catheter shaft 126 of the balloon catheter 116 can be formed from any of various suitable materials, such as nylon, braided stainless steel wires, or a polyether block amide (commercially available as Pebax®). In some examples, the inner and outer shafts 126, 134 can have longitudinal sections formed from different materials in order to vary the flexibility of the shafts along their lengths. In some examples, the inner shaft 134 can have an inner liner or layer formed of Teflon® to minimize sliding friction with a guide wire.

The distal end portion of the guide catheter shaft 122 comprises a steerable section 168 (FIG. 4), the curvature of which can be adjusted by the operator to assist in guiding the delivery apparatus 100 through the patient's vasculature, and in particular, the aortic arch. The handle portion 120 in the illustrated example comprises a distal handle portion 146 and a proximal handle portion 148 (FIG. 3). The distal handle portion 146 functions as a mechanism for adjusting the curvature of the distal end portion of the guide catheter shaft 122 and, in some examples, as a flex indicating device that allows a user to measure the relative amount of flex of the distal end of the guide catheter shaft 122. In addition, the flex indicating device can provide a visual and tactile response at the handle the device, which provides a surgeon with an immediate and direct way to determine the amount of flex of the distal end of the catheter.

The distal handle portion 146 can be operatively connected to the steerable section 168 and can function as an adjustment mechanism to permit operator adjustment of the curvature of the steerable section via manual adjustment of the handle portion. Explaining further, the distal handle portion 146 can comprise a flex activating member 150, an indicator pin, and a cylindrical main body, or housing (FIG. 3). In some examples, the flex activating member 150 can comprise an adjustment knob. One or more pull wires can couple the adjustment knob of the flex activating member 150 to the steerable section 168 (FIG. 4) to adjust the curvature of the steerable section 168 upon rotation of the adjustment knob of the flex activating member 150.

In particular examples, the steerable section 168 in its non-deflected shape is slightly curved and in its fully curved position, the steerable section generally conforms to the shape of the aortic arch. In other examples, the steerable section 168 can be substantially straight in its non-deflected position.

The distal handle portion 146 can have other configurations that are adapted to adjust the curvature of the steerable section 168. One such alternative handle configuration is shown in co-pending U.S. Pat. No. 7,780,723, which is incorporated herein by reference in its entirety. Additional details relating to the steerable section and handle configuration discussed above can be found in U.S. Pat. Nos. 8,568,472 and 9,339,384, both of which are incorporated herein by reference in their entireties.

As described above, when the delivery apparatus 100 is introduced into the vasculature of the patient, a crimped prosthetic valve 10 can be positioned proximal to the balloon 128 (FIG. 5). Prior to expansion of the balloon 128 and deployment of prosthetic valve 10 at the treatment site (or target implantation site), the prosthetic valve 10 can be moved relative to the balloon 128 (or vice versa) to position the crimped prosthetic valve on the balloon 128 for deploying (radially expanding) the prosthetic valve (FIG. 7). The proximal handle portion 148 (FIG. 3) can serve as an adjustment device that can be used to move the balloon 128 proximally into position within the frame of prosthetic valve 10, and further to accurately position the 128 balloon and the prosthetic valve 10 at the desired deployment location.

In some examples, the proximal handle portion 148 can comprise an adjustment mechanism which is configured to adjust the axial position of the outer balloon catheter shaft 126 relative to the guide catheter shaft 122, the adjustment mechanism comprising an adjustment knob 184 (FIG. 3). The adjustment knob 184 can be utilized to position the prosthetic valve 10 on the balloon 128 and/or once the prosthetic valve 10 is on the balloon 128 (or if the valve is already on the balloon), to position the prosthetic valve 10 and the balloon 128 at the desired deployment site within the native valve annulus.

In some examples, the proximal handle portion 148 can further comprise a securement mechanism 198 (FIG. 3) which is configured to retain the position of the outer balloon catheter shaft 126 relative to the proximal handle portion 148 for use of the adjustment knob 184 of the adjustment mechanism. For example, the securement mechanism 198 can be operable to restrain movement of the outer balloon catheter shaft 126 (in the axial and rotational directions) relative to the proximal handle portion 148.

One exemplary method for implanting the prosthetic valve 10 in the native aortic valve is as follows. The prosthetic valve 10 initially can be crimped on a mounting portion (or region) 125 (FIGS. 5 and 6) of the outer balloon catheter shaft 126 immediately adjacent the proximal end of the balloon 128 or slightly overlapping the proximal end of the balloon 128. The proximal end of the prosthetic valve can abut the distal end 140 of the guide catheter shaft 122 (FIG. 5), which keeps the prosthetic valve in place on the balloon catheter shaft 126 as the delivery apparatus 100 and prosthetic valve 10 are inserted through an introducer sheath. The prosthetic valve 10 can be delivered in a transfemoral procedure by first inserting an introducer sheath into the femoral artery and pushing the delivery apparatus 100 through the introducer sheath into the patient's vasculature.

After the prosthetic valve 10 is advanced through the narrowest portions of the patient's vasculature (for example, the iliac artery), the prosthetic valve 10 can be moved onto the balloon 128. For example, a convenient location for moving the prosthetic valve onto the balloon is the descending aorta. The prosthetic valve can be moved onto the balloon 128, for example, by holding the handle portion 120 steady (which retains the guide catheter shaft 122 in place) and moving the outer balloon catheter shaft 126 in the proximal direction relative to the guide catheter shaft 122. As the outer balloon catheter shaft 126 is moved in the proximal direction, the distal end 140 of the guide catheter shaft 122 pushes against the prosthetic valve 10, allowing the balloon 128 to be moved proximally through the prosthetic valve 10 in order to center the prosthetic valve 10 on the balloon 128, as depicted in FIG. 7.

In some examples, the outer balloon catheter shaft 126 can include one or more radiopaque markers to assist the user in positioning the prosthetic valve at the desired location on the balloon 128. The outer balloon catheter shaft 126 can be moved in the proximal direction by simply sliding/pulling the outer balloon catheter shaft 126 in the proximal direction if the securement mechanism 198 is not engaged to retain the outer balloon catheter shaft 126. For more precise control of the outer balloon catheter shaft 126, the securement mechanism 198 can be engaged to retain the outer balloon catheter shaft 126, in which case the adjustment knob 184 is rotated to effect movement of the outer balloon catheter shaft 126 and the balloon 128.

In some examples, as shown in FIG. 6, the delivery apparatus 100 can further include a mounting member 160 secured to the outer surface of the inner shaft 34 within the balloon 128. The mounting member 160 can help retain the prosthetic valve in place on the balloon 128 by facilitating the frictional engagement between the prosthetic valve and the outer surface of the balloon 128. The mounting member 160 can help retain the prosthetic valve in place for final positioning of the prosthetic valve at the deployment location, especially when crossing the native leaflets, which typically are calcified and provide resistance against movement of the prosthetic valve. The mounting member 160 can be configured to allow the inflation fluid (for example, saline) to flow unobstructed from the proximal end of the balloon 128 to the distal end of the balloon 128.

In some examples, as shown in FIGS. 6 and 7, the nose cone 132 can include a proximal portion 162 inside the balloon 128 to assist in positioning the prosthetic valve. The proximal portion 162 can comprise a tapered member that has a maximum diameter at its proximal end adjacent the distal end of the prosthetic valve 10 (FIG. 7) and tapers in a direction toward the distal end of the nose cone 132. The tapered member or the proximal portion 162 can serve as a transition section between the nose cone 132 and the prosthetic valve 10 as the prosthetic valve is pushed through the calcified native leaflets by shielding the distal end of the prosthetic valve from contacting the native leaflets. Although FIG. 7 shows the prosthetic valve 10 having a crimped diameter slightly larger than the diameter of the tapered member of the proximal portion 162 at its proximal end, the tapered member of the proximal portion 162 can have a diameter at its proximal end that is the same as or slightly larger than the diameter of the crimped prosthetic valve, or at least the same as or slightly larger than the diameter of the metal frame of the crimped prosthetic valve.

As shown in FIG. 7, the prosthetic valve can be positioned on the balloon 128 for deployment such that the distal end of the prosthetic valve 10 is slightly spaced from the proximal portion 162 of the nose cone 132. When the prosthetic valve is positioned as shown in FIG. 7, the guide catheter shaft 122 can be moved proximally relative to the outer balloon catheter shaft 126 so that the guide catheter shaft 122 is not covering the inflatable portion of the balloon 128, and therefore will not interfere with inflation of the balloon.

As the prosthetic valve 10 is guided through the aortic arch and into the ascending aorta, the curvature of the steerable section 168 can be adjusted (as explained in detail above) to help guide or steer the prosthetic valve through that portion of the vasculature. As the prosthetic valve is moved closer toward the deployment location within the aortic annulus, it becomes increasingly more difficult to control the precise location of the prosthetic valve by pushing or pulling the handle portion 120 due to the curved section of the delivery apparatus. When pushing or pulling the handle portion 120, slack can be removed from the curved section of the delivery apparatus before the pushing/pulling force is transferred to the distal end of the delivery apparatus. Consequently, the prosthetic valve tends to “jump” or move abruptly, making precise positioning of the prosthetic valve difficult.

For more accurate positioning of the prosthetic valve within the aortic annulus, the prosthetic valve 10 can be placed as close as possible to its final deployment location (for example, within the aortic annulus such that an inflow end portion of the prosthetic valve is in the left ventricle and an outflow end portion of the prosthetic valve is in the aorta) by pushing/pulling the handle portion 120, and final positioning of the prosthetic valve can be accomplished using the adjustment knob 184 (FIG. 3). To use the adjustment knob 184, the securement mechanism 98 can be placed in a locked position. Then, the handle portion 120 is held steady (which retains the guide catheter shaft 122 in place) while rotating the adjustment knob 184 to move the outer balloon catheter shaft 126, and thus the prosthetic valve 10, in the distal or proximal directions. For example, rotating the adjustment knob 184 in a first direction (for example, clockwise), moves the prosthetic valve proximally into the aorta, while rotating the knob 184 in a second, opposite direction (for example, counterclockwise) advances the prosthetic valve distally toward the left ventricle. Advantageously, operation of the adjustment knob 184 is effective to move the prosthetic valve in a precise and controlled manner without sudden, abrupt movements as can happen when pushing or pulling the delivery apparatus 100 for final positioning.

When the prosthetic valve 10 is at the deployment location, the balloon 128 is inflated to expand the prosthetic valve 10 so as to contact the native annulus. The expanded prosthetic valve (for example, as shown in FIG. 1) becomes anchored within the native aortic annulus by the radial outward force of the valve's frame against the surrounding tissue.

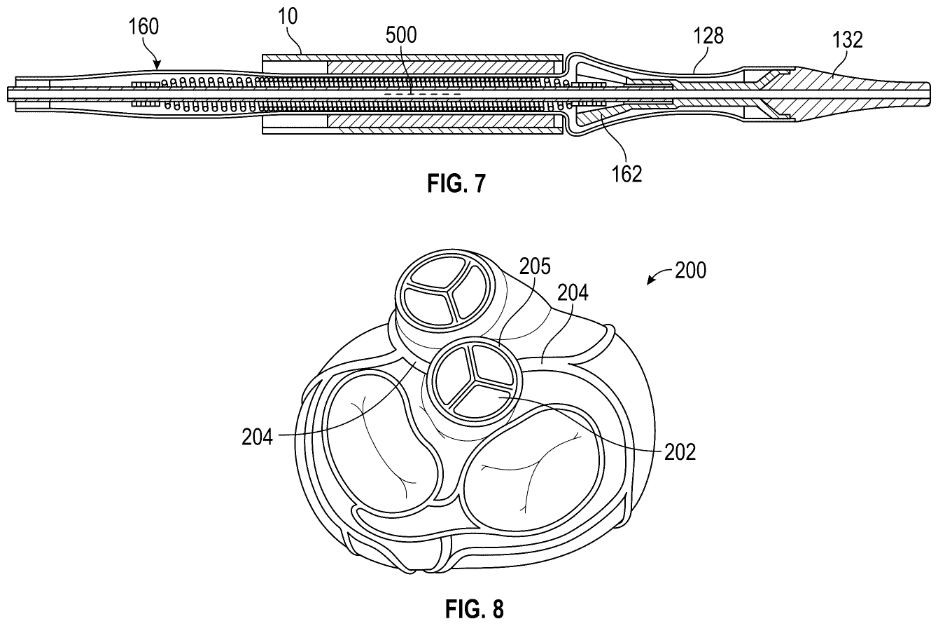

In some examples, the delivery apparatus 100 (or another, similar delivery apparatus) can be configured to deploy and implant a prosthetic heart valve (for example, prosthetic valve 10 of FIG. 1 or prosthetic heart valve 50 of FIGS. 2A and 2B) in the native aortic annulus of a native aortic valve. An exemplary heart 200 including an aortic valve 202 is shown in FIG. 8. As shown in FIG. 8, two coronary arteries (for example, the left coronary artery and the right coronary artery) 204 are coupled to and branch off from the aorta 205, proximate to the aortic valve 202. The coronary arteries 204 carry oxygenated blood from the aorta to the muscle of the heart 200.

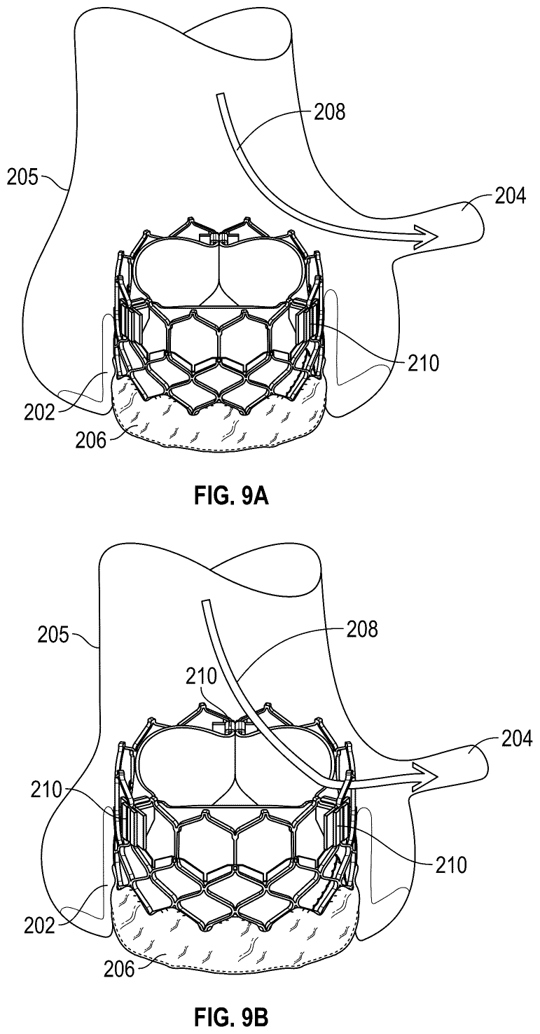

As shown in FIG. 9A, since the prosthetic heart valve 206 is implanted in the native aortic annulus of the aortic valve 202, blood flow may exit the prosthetic heart valve 206, flow into the aorta 205, and then flow over top of the outflow end of the prosthetic heart valve 206 and/or through open cells (for example, open cells that are not constantly covered by leaflets of the prosthetic heart valve) in the frame of the prosthetic heart valve 206, to the coronary artery 204 (only one shown in FIGS. 9A and 9B). Depending on a patient's anatomy, the prosthetic heart valve may cover (for example, be placed in front of) at least a portion of the opening to the coronary artery 204, as shown in the example depicted in FIG. 9B. In some examples, a subsequent coronary procedure that requires a device (for example, catheter) to access the coronary arteries 204 may be required, after implanting the prosthetic heart valve 206 (where the device may require access to the coronary arteries 204 via the exemplary paths 208 shown in FIGS. 9A and 9B). If a commissure 210 of the prosthetic heart valve 206 is arranged in front of (or adjacent to) an opening to one of the coronary arteries 204 (FIG. 9B), access to the coronary arteries 204 may be at least partially blocked. For example, since adjacent leaflets are coupled together at the commissures 210, the commissures 210 can make it more difficult for a coronary access device to pass through the open cells of the prosthetic heart valve 206 in order to reach the coronary arteries 204.

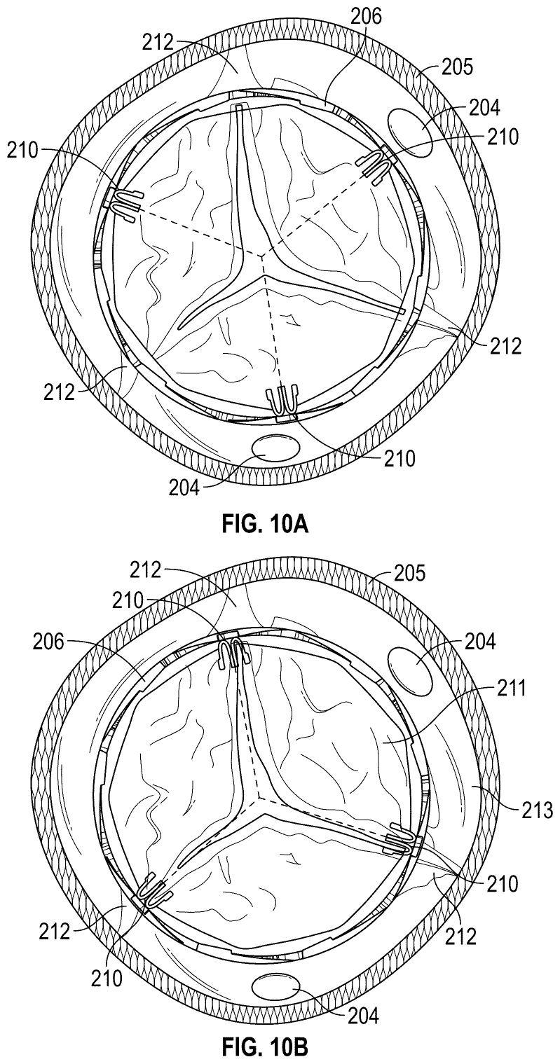

Thus, instead of deploying the prosthetic heart valve with the delivery apparatus in a random rotational orientation relative to the aorta 205, which may result in commissures 210 of the prosthetic heart valve 206 being arranged in front of the coronary arteries 204 (as shown in FIG. 10A), it may be desirable to deploy the prosthetic heart valve 206 in a targeted rotational orientation where the commissures 210 are positioned away from and do not block the coronary arteries 204 (as shown in FIG. 10B). For example, as shown in FIG. 10B, the delivery apparatus can be configured to enable a user (for example, surgeon) operating the delivery apparatus to deploy the prosthetic heart valve 206 such that cusps 211 of the radially expanded prosthetic heart valve 206 are circumferentially aligned with native cusps 213 of the aortic valve 202, thereby aligning the commissure 210 of the prosthetic heart valve 206 with the native commissures 212 of the aortic valve 202.

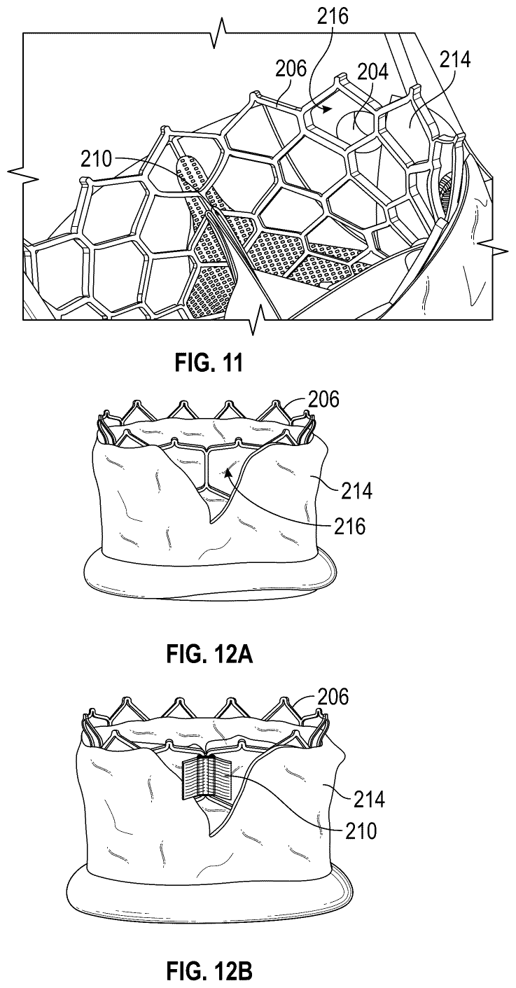

As explained further below, the delivery apparatus can be configured such that the user can visualize, under fluoroscopic imaging (or fluoroscopy), the rotational positioning of the prosthetic heart valve 206 relative to the native valve. This may enable a user to rotate the prosthetic heart valve 206 into a desired rotational position to achieve the cusp and commissure alignment shown in the example of FIG. 10B, thereby enabling easier access to the coronary arteries 204 through the implanted prosthetic heart valve 206 via a coronary access device. Additionally, this positioning of the prosthetic heart valve can facilitate a later, leaflet cutting procedure that provides increased blood flow to the coronary arteries, as shown in FIGS. 11-12B.

For example, as shown in FIG. 11, a native leaflet 214 of the native valve (for example, aortic valve 202) can be split (or cut) longitudinally (relative to a central longitudinal axis of the prosthetic heart valve 206) at a location of an entrance to a coronary artery 204. This enables increased blood flow and/or a coronary access device to enter the coronary artery 204 from the aorta, through one or more open (for example, not covered by leaflets) cells 216 of the prosthetic heart valve 206.

As shown in FIG. 12A, splitting a native leaflet 214 (a leaflet of a host surgical prosthetic valve, as shown in FIGS. 12A and 12B) at a region of a frame of the prosthetic heart valve 206 that is between two adjacent commissures 210 results in open cells 216 that can receive blood flow and coronary access devices therethrough. However, as shown in FIG. 12B, splitting the native leaflet 214 in a region of the frame of the prosthetic heart valve 206 that includes the commissure 210 (for example, due to the commissure 210 being positioned in front of the entrance to the coronary artery 204), does not result in open cells 216 being arranged in front of the entrance to the coronary artery 204. Instead, the commissure 210 can continue to block access to the coronary artery 204.

Thus, it is desirable for a user operating the delivery apparatus to be able to determine during an implantation procedure where the cusps (and/or commissures) of the prosthetic heart valve are located relative to the native anatomy (for example, the native leaflet cusps of the native valve). During an implantation procedure, fluoroscopy (for example, long axis fluoroscopy) can be used to visualize the distal end portion of the delivery apparatus, including the radially compressed prosthetic valve, relative to the surrounding native anatomy (for example, the native aortic valve). An exemplary fluoroscopic image 300 of a native (for example, aortic) valve 302 viewed with long axis fluoroscopy in a standard, three-cusp imaging view is shown in FIG. 13. As shown in FIG. 13, the native aortic valve 302 includes three leaflets (or cusps): the non-coronary cusp 304, the right coronary cusp 306, and the left coronary cusp 308. In the three-cusp view, the non-coronary cusp 304 and the left coronary cusp 308 are arranged opposite one another in the view and are each overlapped by a portion of the right coronary cusp 306 (which is anteriorly positioned in the imaging view). The leaflets of the native aortic valve 302 are separated from one another by native commissures 310, indicated schematically in FIG. 13 with dots. However, the imaging view provided by long axis fluoroscopy does not allow a user to easily visualize the location of these cusps and commissures, especially their 3D, circumferential orientation relative to one another and within the fluoroscopic image 300.

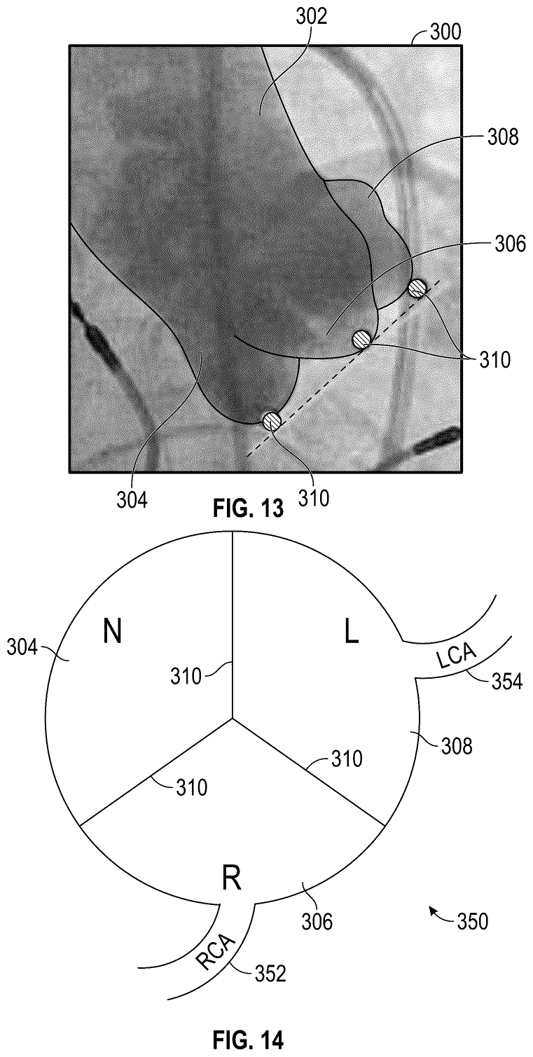

In contrast, computed tomography (CT) imaging (or another type of 3D imaging) can be used to visualize the circumferential orientation of the commissures 310 and cusps of the native aortic valve 302, around a circumference of the native aortic valve 302. FIG. 14 shows a schematic of an exemplary view 350 of the native aortic valve 302 that can be obtained with short axis CT imaging. As shown in the view 350 of FIG. 14, a circumferential position of the commissures 310 between each of the non-coronary cusp 304, the right coronary cusp 306, and the left coronary cusp 308 can be visualized. Further, FIG. 14 identifies a location of the right coronary artery 352 and a location the left coronary artery 354, each of which is offset (circumferentially) from adjacent commissures 310. Thus, as explained above, it is desirable for the prosthetic heart valve to be implanted such that its cusps and commissures are circumferentially aligned with the native valve cusps and commissure 310, so as to avoid blocking access to the right coronary artery 352 and the left coronary artery 354.

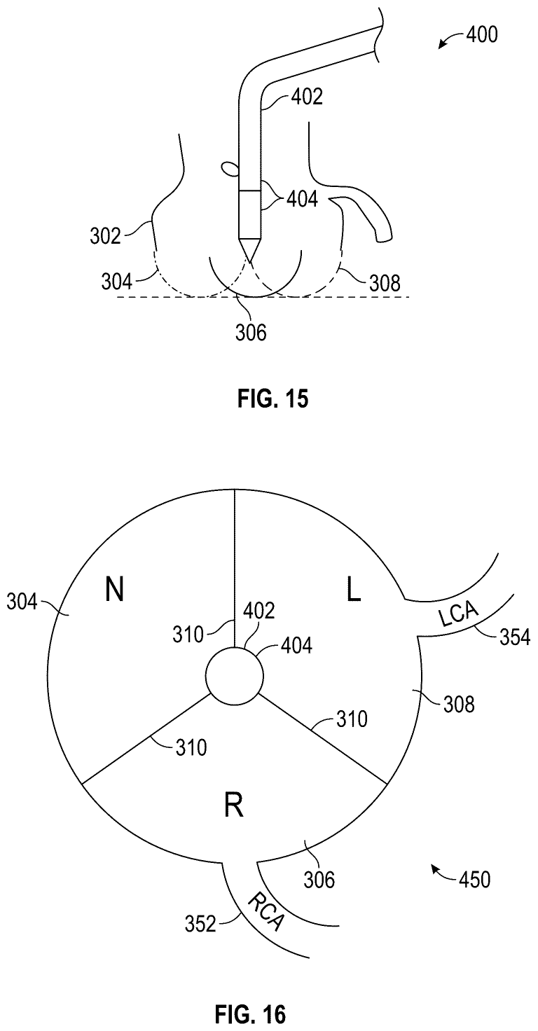

However, during a prosthetic heart valve implantation procedure, short axis CT imaging is not available and an image such as that shown in FIG. 14 cannot be obtained. At best, a user can obtain a fluoroscopic image obtained by long axis fluoroscopy (for example, the fluoroscopic image 300 of FIG. 13). An exemplary schematic of a fluoroscopic image 400 that can be obtained during a prosthetic heart valve implantation procedure is shown in FIG. 15. The fluoroscopic image 400 shows an axial position of the distal end portion of a delivery apparatus 402, and a radially compressed prosthetic heart valve 404, relative to the native aortic valve 302. The prosthetic heart valve 404 can be mounted on/around a portion of the distal end portion of the delivery apparatus 402. In some examples, when the distal end portion of the delivery apparatus 402 is disposed proximate to the annulus of the native aortic valve 302 (as shown in FIG. 15), the prosthetic heart valve 404 can be disposed around an inflatable balloon of the delivery apparatus 402.

Further, even if (for the sake of argument) a short axis CT image were available during the valve implantation procedure, it would not be possible to visualize the circumferential position of the commissures of the prosthetic heart valve 404, as shown in the exemplary CT image 450 of FIG. 16. Since the commissures of the radially compressed prosthetic heart valve 404 may not be visible and differentiable from a remainder of the prosthetic heart valve 404 (the frame of the prosthetic heart valve) under such imaging, particularly with the available long axis fluoroscopy, a user operating the delivery apparatus would still be unable to orient the delivery apparatus and the radially compressed prosthetic heart valve such that commissures of the prosthetic heart valve are in alignment with the commissures 310 of the native aortic valve 302 upon deployment and implantation of the prosthetic heart valve within the annulus of the native aortic valve 302.

To address these issues with commissure and cusp alignment between the native valve and the prosthetic heart valve, one or more radiopaque markers that are visible under medical imaging can be incorporated into the distal end portion of the delivery apparatus. At least one radiopaque marker can correspond to a specified location on the prosthetic heart valve, such as a selected cusp of the prosthetic heart valve. Thus, the delivery apparatus can include at least one and up to three radiopaque markers, at least one marker (and in some examples, all three markers) corresponding to a circumferential location of a middle of a corresponding cusp of the prosthetic heart valve. As a result, when the prosthetic heart valve is radially expanded by the delivery apparatus, a middle of the selected cusp of the prosthetic heart valve can be located at the same circumferential position as the corresponding radiopaque marker on the delivery apparatus. As described further below, the delivery apparatus can include one, two, or three radiopaque markers, where one or more of the markers correspond to the location of a middle of one or more corresponding cusps of the prosthetic heart valve. As such, in the case of multiple markers, the markers can be spaced 180 degrees (for two markers) or 120 degrees (for three markers) apart from one another around the delivery apparatus. The markers can have various shapes and sizes that are distinguishable in the fluoroscopic image, as described further below.

In some examples, the delivery apparatus can include two radiopaque markers 500 that are disposed 180 degrees apart from one another around a circumference of the delivery apparatus. For example, as shown in the schematic of the exemplary fluoroscopic image 502 of FIG. 17 and the schematic of the exemplary short axis CT image 510 of FIG. 18, two radiopaque markers 500 that are arranged 180 degrees apart from one another around the circumference of the delivery apparatus 402 can be visualized under medical imaging (only one marker 500 is visible in FIG. 17 since the two markers are overlapping in the imaging view, as explained further below).

As an example, during an implantation procedure, the fluoroscopic image 502 of FIG. 17 may be available and allow a user to visualize the markers 500 on the distal end portion of the delivery apparatus 402, relative to the native anatomy. When the distal end portion of the delivery apparatus 402, including the radially compressed prosthetic heart valve 404 mounted thereon, is at or proximate to the native aortic valve 302 (as shown in FIG. 17), the distal end portion of the delivery apparatus 402, and thus the radially compressed prosthetic heart valve 404, can be rotated until the two radiopaque markers 500 overlap one another in the fluoroscopic imaging view, thereby indicating one of the two markers 500 is positioned at an anterior (front) of the imaging view (as shown in FIG. 17). The markers 500 can be positioned on the delivery apparatus 402 such that when the two markers 500 overlap one another in the imaging view of the fluoroscopic image 502, the cusps of the prosthetic heart valve 404 will be circumferentially aligned with the native cusps 304, 306, and 308 of the native aortic valve 302, when deployed from the delivery apparatus 402 and implanted in the native annulus of the native aortic valve 302. More specifically, when the two markers 500 overlap and appear to be aligned with a middle of the right coronary cusp 306, which is at a front of the fluoroscopic image 502, a corresponding cusp of the prosthetic heart valve 404 will overlap the right coronary cusp 306 when the prosthetic heart valve 404 is deployed.

For example, FIG. 18 shows what the delivery apparatus 402 and the two markers 500 would look like in a short axis CT image, for the same position of the delivery apparatus shown in FIG. 17 (even though such a CT image is not available during a valve implantation procedure). As shown in FIG. 18, the markers 500 are disposed 180 degrees apart from one another around the circumference of the delivery apparatus 402. Thus, when the two markers 500 are overlapping and aligned with the middle of the right coronary cusp 306 in the available fluoroscopic image 502 of FIG. 17, one of the markers 500 (the anterior marker) will be circumferentially aligned with the right coronary cusp 306 and another one of the markers 500 (the posterior marker) will be circumferentially aligned with one of the commissures 310 of the native aortic valve 302 (as illustrated in FIG. 18), and thus, the prosthetic heart valve 404 can be intentionally implanted with cusps and commissures in alignment with the native cusps and commissures 310 of the native aortic valve 302.

The two markers 500 can be configured with a different shape, size, or longitudinal alignment such that are distinguishable in the imaging view and a user can identify which of the two markers 500 is anterior and posterior within the imaging view. The user can then rotate the delivery apparatus until the two markers 500 are overlapping in the imaging view and the anterior marker is aligned with the right coronary cusp (or alternate specified cusp of the native valve). In this way, a pair of radiopaque markers 500 on the delivery apparatus can enable a user to orient the delivery apparatus using a standard long axis fluoroscopic imaging view such that the prosthetic heart valve is implanted in the native valve annulus with cusps aligned with cusps of the native valve.

The markers 500 and additional marker examples described herein can be configured to be visible under medical imaging. For example, the markers 500 can comprise a radiopaque material that is configured to be visible under medical imaging, such as fluoroscopy and/or other types of X-ray imaging. In some examples, the markers 500 can comprise a radiopaque or other material that is configured to be visible under MRI, ultrasound, and/or echocardiogram

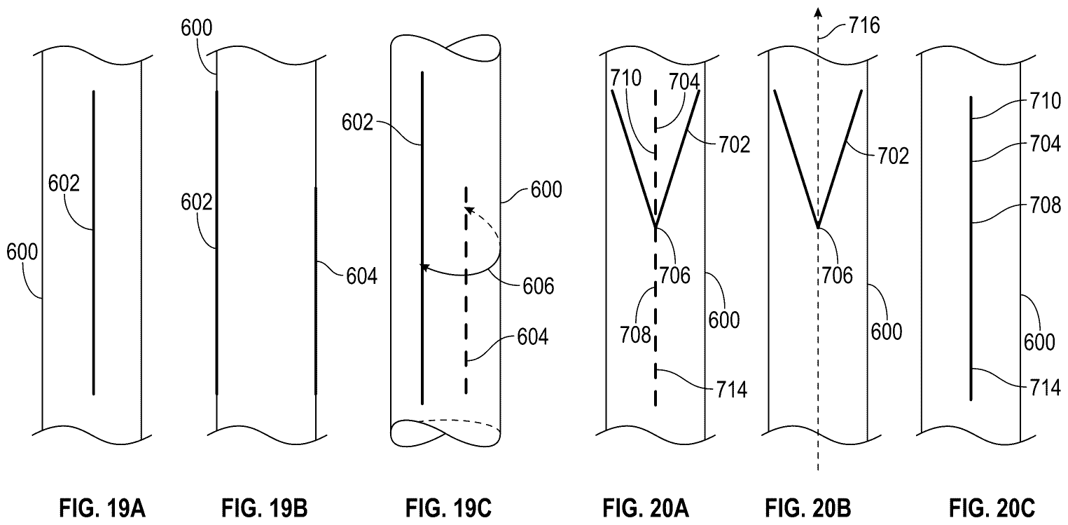

Exemplary configurations for the pair of markers 500 are shown in FIGS. 19A-20C. Specifically, FIGS. 19A-19C illustrate one example of a pair of radiopaque markers, including a first marker 602 and a second marker 604, that can be mounted on or embedded within a shaft 600 of a delivery apparatus (for example, delivery apparatus 100), 180 degrees apart from one another around a circumference of the shaft 600. As shown in FIGS. 19A-19B, the first marker 602 and second marker 604 are linear markers (for example, lines). In some examples, as shown in FIGS. 19B and 19C, the first marker 602 and the second marker 604 can have different lengths (the second marker 604 is shown shorter than the first marker 602 in FIGS. 19B and 19C). In alternate examples, the first marker 602 and the second marker 604 can have different widths, different lengths and widths, different longitudinal positions (one higher than the other in the axial direction, for example), or the like.

FIG. 19A is a side view depicting a first rotational orientation of the shaft 600, where the first marker 602 and second marker 604 are aligned and overlap one another, thereby appearing as a single line. FIG. 19B is another side view depicting a second rotational orientation of the shaft 600, where the first marker 602 and second marker 604 are on opposite sides of the shaft 600 (due to their 180-degree separation). FIG. 19C is a perspective view of the shaft 600 depicting the first marker 602 as a solid line (since it is at the front of the view) and the second marker 604 as a dashed line (since it is at the back of the view). The positioning of the first marker 602 and the second marker 604, 180 degrees apart around the circumference of the shaft 600 is illustrated by angle 606 in FIG. 19C. As the shaft 600 is rotated, a user can see the first marker 602 and the second marker 604 moving relative to one another and determine their relative positions in the imaging view (for example, anterior vs. posterior).

As explained further below with reference to FIGS. 4-7, the shaft 600 can be one of the shafts of the delivery apparatus 100, such as a distal end portion of one of the shafts of the balloon catheter 116 (such as outer shaft 126 or inner shaft 134).

FIGS. 20A-20C illustrate another example of a pair of radiopaque markers, including a first marker 702 and a second marker 704, that can be mounted on or embedded within the shaft 600 of the delivery apparatus (for example, delivery apparatus 100), 180 degrees apart from one another around a circumference of the shaft 600. Further, the first marker 702 and second marker 704 can be aligned within one another in the axial direction (for example, are arranged on the shaft 600 at a same axial position such that at least a portion of the first marker 702 and second marker 704 overlap one another in the fluoroscopic imaging view, as explained herein). FIG. 20A is a transparent, side view of the shaft 600 showing the first marker 702 in solid lines (on a front side of the shaft 600) and the second marker 704 in dashed lines (on a back side of the shaft 600, 180 degrees away from the first marker 702). FIG. 20B is a side view of a first side of the shaft 600 showing the first marker 702 and FIG. 20C is a side view of an opposite, second side of the shaft 600 showing the second marker 704.

The first marker 702 is V-shaped (or shaped as a “V”) and the second marker 704 is a linear marker (for example, shaped as a line). As shown in FIG. 20A, when the first marker 702 and second marker 704 are aligned and overlap one another in the view, an apex 706 of the V-shaped first marker 702 overlaps a central portion 708 of the linear second marker 704 and a first end portion 710 of the second marker 704 is positioned between the two arms 712 of the “V” of the first marker 702. In other examples, the apex 706 of the first marker 702 can be positioned more distal or proximal on the shaft 600 relative to the second marker 704, thereby causing the apex 706 to overlap the first end portion 710 or a second end portion 714 of the second marker 704 in the view of FIG. 20A. In some examples, the arms 712 of the first marker 702 can be shorter than shown in FIGS. 20A and 20B and not extend to the end of the second marker 704. In still other examples, the arms 712 of the first marker 702 can have different lengths and/or the first marker 702 can only have one arm, thereby having asymmetry that allows a user to identify an anterior or posterior position of the first marker 702 in the imaging view.

In alternate examples, instead of being V-shaped, the first marker 702 can have an asymmetric shape with a discernable axis or connecting point that is configured to align with and overlap the linear second marker 704 in the imaging view, such as an E-shape, half Y-shape (for example, only one arm of the top of the Y), a P-shape, a greater or less than sign (“>”) that is meant to connect on one side with the linear second marker 704, or the like. For example, the first marker 702 can have a portion that is configured to extend outward to one side of a longitudinal axis 716 of the first marker 702 that is configured to align with the linear second marker 704.

The shaft (for example, shaft 600) of the delivery apparatus on which the radiopaque markers (for example, markers 500, 602 and 604, 702 and 704, 800, or 900, as described herein) can be mounted on or embedded within can be one of the shafts of the balloon catheter 116 of the delivery apparatus 100 (FIGS. 3-7). For example, FIGS. 4-7 depict exemplary locations on the distal end portion of the delivery apparatus 100 that the radiopaque markers can be located. In FIGS. 4-7, one marker of one, two, or three radiopaque markers of the selected shaft of the delivery apparatus is illustrated by a dashed line and the other marker(s) is not visible (due to them being arranged 180 or 120 degrees away from the first marker, and thus, behind the delivery apparatus in the side views of FIGS. 4-7). Using a dashed line for the marker 500 is for ease of illustration only, to differentiate from the other lines of the delivery apparatus and does not necessarily represent the actual configuration of the marker (for example, the marker can be a solid line, as shown in FIGS. 19A-20C, or another shape, as described further herein).

For example, in one instance, the radiopaque markers 500 (or any of the other radiopaque markers described herein) can be mounted on or embedded within the outer balloon catheter shaft 126 of the balloon catheter 116 (FIGS. 4 and 6). For example, in some instances, as shown in FIG. 6, the radiopaque markers 500 can be mounted on or embedded within a distal end of the outer balloon catheter shaft 126. In some examples, the distal end of the outer balloon catheter shaft 126 can be part of the mounting portion 125 (FIG. 6), and thus, the markers 500 can be disposed underneath the radially compressed valve 10.

In another example, the radiopaque markers 500 can be mounted on or embedded within the inner shaft 134 of the balloon catheter 116. For example, as shown in FIGS. 5-7, the radiopaque markers 500 can be mounted on or embedded within the inner shaft 134, distal to the outer balloon catheter shaft 126. In some examples, the radiopaque markers 500 can be disposed on a distal end portion of the inner shaft 134 which is arranged inside the balloon 128 (FIG. 6) and can be disposed underneath the prosthetic heart valve 10 when the prosthetic heart valve 10 is disposed over the balloon 128 and ready to be deployed from the delivery apparatus 100 (FIG. 7).

In other examples, the radiopaque markers 500 can be disposed on a distal end portion of the inner shaft 134 which is arranged proximate to the distal end of the outer balloon catheter shaft 126 (in some examples, at the valve mounting portion 125).

It should be noted that each of the dashed lines representing the radiopaque markers 500 in FIGS. 4-7 illustrate one exemplary axial location (relative to the central longitudinal axis 170) for the radiopaque markers 500. In this way, in some examples, the delivery apparatus 100 may only include one pair of radiopaque markers, a single radiopaque marker, or three radiopaque markers (for example, at one location along the distal end portion of the delivery apparatus, even though multiple locations are shown, for example in FIG. 6, for illustrative purposes only). Further, a length of the dashed lines representing the radiopaque markers 500 in FIGS. 4-7 are not meant to be limiting. For example, the markers 500 can have various lengths, widths, and shapes, as described herein, and in some examples, the markers 500 can be shorter, a same length, or longer than a length of the radially compressed prosthetic heart valve.

As disclosed above with reference to FIGS. 3-7, the prosthetic valve 10 on the delivery apparatus 100 can be positioned axially at the deployment or implantation site via adjusting one or more adjustment mechanisms (for example, knobs) of the handle portion 120. The exemplary method for deploying and implanting the prosthetic valve described above can further include rotating the radially compressed prosthetic valve by rotating a proximal end portion of the delivery apparatus 100, in order to position the prosthetic heart valve in a selected rotational position at the implantation site (or relative to the native anatomy). For example, an entire distal end portion of the delivery apparatus 100, including the balloon catheter 116 and the guide catheter 144 can be rotated together by rotating the handle portion 120.

Alternatively, by unlocking the balloon catheter 116 and the guide catheter 114 (for example, via the securement mechanism 198) such that the balloon catheter 116 can move independent of the guide catheter 114, the distal end portion of the balloon catheter 116, including the prosthetic valve mounted thereon, can be rotated by rotating the proximal end portion of the balloon catheter 116 (disposed proximal to the handle portion 120). For example, the shaft of the balloon catheter on which the pair of radiopaque markers 500 are disposed can be rotated relative to the handle portion 120. In this way, upon reaching the implantation site, the distal end portion of the balloon catheter 116 can be rotated, thereby rotating the outer balloon catheter shaft 126, the inner shaft 134, the balloon 128, and the prosthetic valve (for example, valve 10 shown in FIGS. 5 and 7) together, until the pair of radiopaque markers 500 overlap one another and are aligned in the fluoroscopic imaging view (for example, as shown schematically in FIG. 17). The prosthetic heart valve can then be deployed and radially expanded by inflating the balloon, thereby implanting the prosthetic valve with commissures circumferentially aligned with native commissures of the native valve.

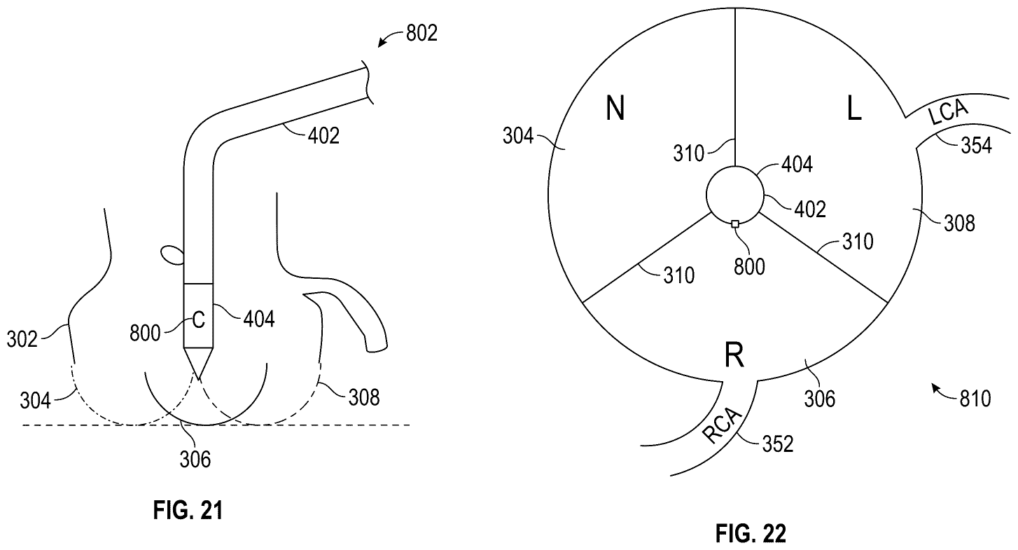

In some examples, instead of two radiopaque markers, the delivery apparatus can include a single radiopaque marker 800 that is reflection asymmetric about its longitudinal axis. For example, FIG. 21 shows the marker 800 having a C-shape. However, in alternate examples, the single marker can have another asymmetric shape, such as an E-shape, P-shape, an arrow shape, a greater than sign (>) or less than sign (<) shape, or the like. Similar to FIGS. 17 and 18, FIGS. 21 and 22 show the marker 800 disposed on a distal end portion of the delivery apparatus 402, as seen in an exemplary fluoroscopic image 802 (FIG. 21) and the schematic image 810 (FIG. 22). The asymmetric marker 800 can be configured such that it is oriented in a certain way (for example, is in a predetermined orientation of two possible readable orientations, such as reading as a forward “C”) when the marker 800 is disposed anteriorly or at a front of the fluoroscopic image 802 (as shown in FIG. 21). Thus, during an implantation procedure, the delivery apparatus 402 can be rotated until the asymmetric marker 800 appears in its forward or preselected orientation that indicates the anterior positioning (“C” in FIG. 21) and is aligned with a middle of the specified cusp of the native valve (the right coronary cusp 306), as shown in FIGS. 21 and 22.

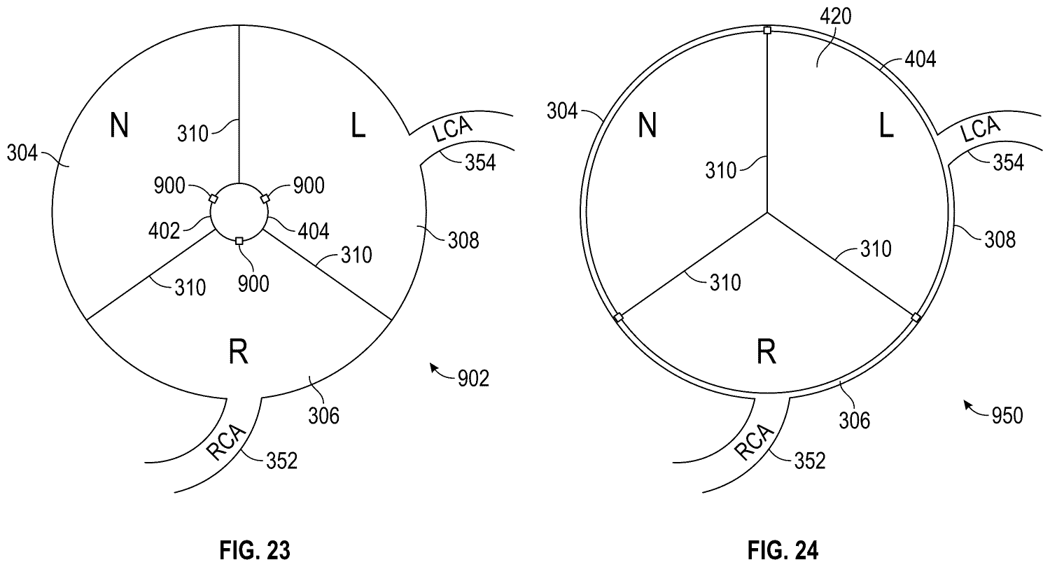

In still other examples, instead of one or two radiopaque markers, the delivery apparatus can include three radiopaque markers 900 that are spaced 120 degrees apart around a circumference of the delivery apparatus, as shown in the exemplary schematic image 902 of FIG. 23. In some examples, each of the three radiopaque markers 900 can be asymmetric markers (similar to the single marker 800). In some instances, the three radiopaque markers 900 can all have different shapes (for example, a C, E, and T shape). In other instances, the three radiopaque marker 900 can all have the same asymmetric shape (for example, three C's). In other examples, two of the three radiopaque markers 900 can be asymmetric while one of the radiopaque markers 900 is symmetric (for example, a line). By incorporating three radiopaque markers 900 on the delivery apparatus 402 that each correspond to one of the cusps of the prosthetic heart valve 404, the amount the delivery apparatus has to be rotated to align the markers 900 with the native valve cusps can be reduced, thereby making the implantation process easier and causing less irritation to the native anatomy (for example, by rotating the prosthetic heart valve by a smaller amount). For example, during an implantation procedure, the marker 900 that is nearest the selected native cusp (for example, the right coronary cusp 36) in the imaging view can be used when rotating the delivery apparatus 402 to align the marker 900 with the middle of the selected native cusp. As a result, alignment can be quicker with a reduced amount (degree) of rotation.

By incorporating a one or more radiopaque markers on the delivery apparatus 402, at least one of the markers corresponding to a location of a middle of cusp of the prosthetic heart valve 404, a user can more easily position the prosthetic heart valve 404 in a target rotational (or circumferential) position at the implantation site relative to the native anatomy. For example, a user utilizing fluoroscopy during an implantation procedure can more easily visualize the distal end portion of the delivery apparatus 402 within the imaging view and rotationally position the distal end portion of the delivery apparatus 402 and the prosthetic heart valve 404 such that at least one of the markers aligns with a middle of one of the native cusps (of the native heart valve). As a result, when the prosthetic heart valve 404 is radially expanded, as shown in the exemplary schematic 950 of FIG. 24, the cusps 420 of the prosthetic heart valve 404 are aligned with the cusps 304, 306, and 308 of the native heart valve. Consequently, commissures of the prosthetic heart valve are spaced away from the openings to the coronary arteries 352, 354, thereby enabling access to the coronary arteries for future interventions.

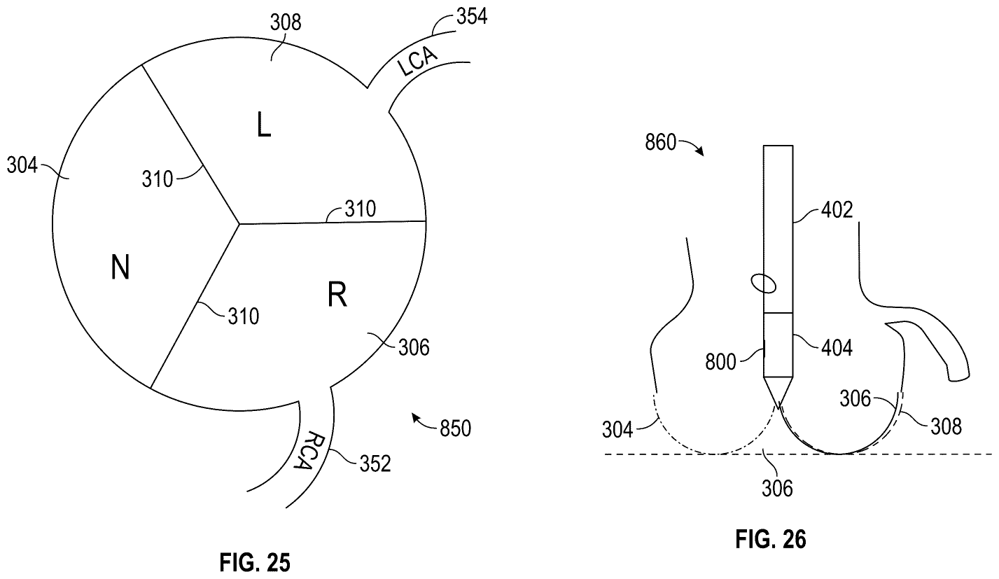

In some examples, a different fluoroscopic imaging view can be selected and used for alignment of the one or more radiopaque markers of the delivery apparatus with the native cusps of the native heart valve. For example, FIG. 26 shows an exemplary fluoroscopic image of a cusp overlap imaging view 860 (or the right/left cusp overlap view) and FIG. 25 shows an exemplary image 850 showing a 3D arrangement of the cusps 304, 306, and 308 in the cusp overlap view. In the cusp overlap imaging view 860, the left coronary cusp 308 and the right coronary cusp 306 overlap one another and the non-coronary cusp 304 is offset from the left coronary cusp 308 and the right coronary cusp 306. When using the cusp overlap imaging view 860 for cusp alignment, the delivery apparatus 402 can be rotated until one of the radiopaque markers, such as the marker 800, appears on a far left side of the imaging view at a middle of the non-coronary cusp 304. For example, when the marker 800 is C-shaped, the marker 800 will appear as a straight line on the left side of the imaging view when it is aligned with the middle of the non-coronary cusp 304, as shown in FIG. 26. The prosthetic heart valve 404 can then be radially expanded and implanted with cusps aligned with the native cusps 304, 306, and 308. Any of the marker combinations described herein can be used with the cusp overlap imaging view 860.

As another example, when the marker(s) are a pair of markers, such as the pair of markers 500 shown in FIGS. 17-18 or markers 602 and 604 shown in FIGS. 19A-19C, the delivery apparatus can be rotated until the two markers appear 180 degrees apart from one another on opposite sides of the cusp overlap imaging view (for example, one on the far left and the other on the far right).

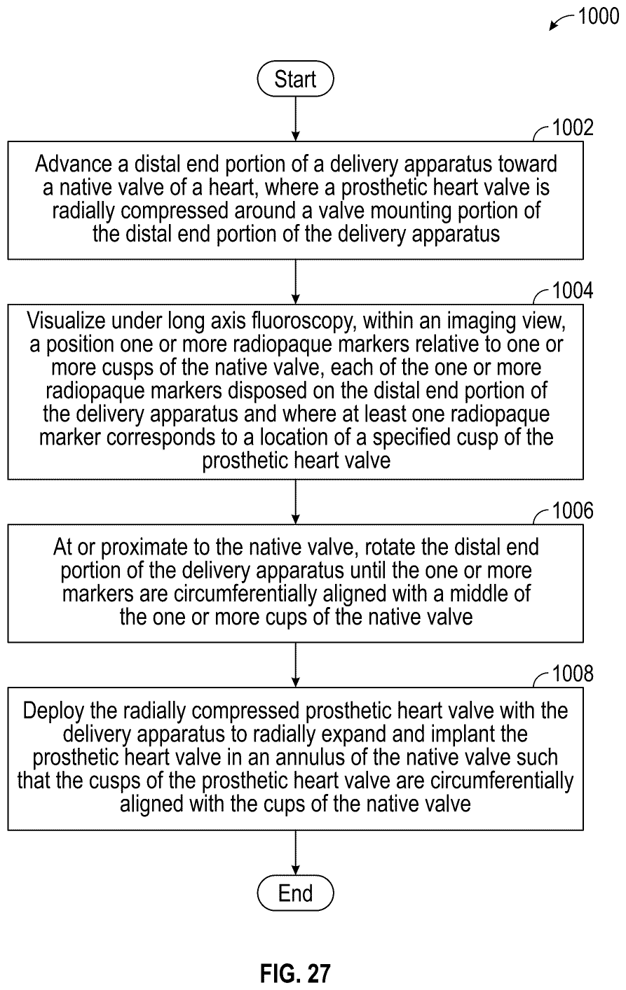

A method for implanting a prosthetic heart valve with cusps circumferentially aligned with the native cusps of the native heart valve using any combination of the one or more markers and imaging views disclosed herein is shown at FIG. 27. Method 1000 begins at 1002 by advancing a distal end portion of a delivery apparatus toward a native valve of a heart, where a prosthetic heart valve is radially compressed around a valve mounting portion of the distal end portion of the delivery apparatus (for example, as shown in FIGS. 7 and 15). As described above, the delivery apparatus can include one or more radiopaque markers (for example, one, two, or three) embedded in or mounted on a distal end portion of a shaft of the delivery apparatus. In some examples, the radiopaque markers can be one or more of the radiopaque markers 500, 602, 604, 702, 704, 800, and/or 900 described herein.

At 1004, the method can include visualizing under long axis fluoroscopy, within an imaging view, a position of the one or more radiopaque markers of the delivery apparatus relative to one or more cusps of the native valve. At least one of the one or more radiopaque markers can correspond to a location of a specified cusp of the prosthetic heart valve. For example, in the case of a single radiopaque marker (for example, marker 800), the marker can correspond (or relate to) a location of a middle of a first cusp of the cusps of the prosthetic heart valve. In the case of two radiopaque markers (for example, markers 500), a first marker of the two markers can correspond to a location of a middle of a first cusp of the cusps of the prosthetic heart valve while the second marker corresponds to a commissure of the prosthetic heart valve. In the case of three radiopaque markers (for example, markers 900), the first, second, and third markers can correspond to a location of a middle of a first, second, and third cusp, respectively, of the cusps of the prosthetic heart valve. As described above, the imaging view can be a three-cusp view, a cusp overlap view, or another selected fluoroscopic imaging view.

In some examples, the method at 1004 can include, first obtaining a 3D image of the patient's heart (for example, via CT or another 3D imaging modality) and selecting a long axis fluoroscopic imaging view, based on the 3D image, that positions a specified cusp of a native valve of the heart in a known location in the selected imaging view. In some instances, the specified cusp can be a right coronary cusp and the known location in the selected imaging view can be a center (and front) of the imaging view. For example, the obtained CT image can allow a user to find a fluoroscopic imaging view that positions the right coronary cusp directly in front of the left non-commissure of the native valve. Further, the selected imaging view can position the middle of the right coronary cusp front and center in the selected imaging view (anterior).

The method continues to 1006 and includes, at or proximate to the native valve, rotating the distal end portion of the delivery apparatus until the one or more markers are circumferentially aligned with a middle of the one or more cusps of the native valve. For example, the rotating at 1006 can include rotating the distal end portion of the delivery apparatus until one marker of the one or more radiopaque markers is aligned with a middle of the specified cusp of the native valve in the known location in the selected imaging view.

In some instances, the imaging view is a three-cusp imaging view where a right coronary cusp of the native valve disposed in a center of the imaging view, and the rotating at 1006 includes rotating the distal end portion of the delivery apparatus until one marker of the one or more markers appears to be positioned anteriorly in the imaging view and is aligned with the middle of the right coronary cusp.

In some instances, the imaging view is a cusp overlap view where a non-coronary cusp of the native valve is disposed on a left side of the imaging view while a right coronary cusp and left coronary cusp of the native valve are superimposed with one another on a right side of the imaging view, and the rotating at 1006 includes rotating the distal end portion of the delivery apparatus until one marker of the one or more markers appears on a far left side of the imaging view at a middle of the non-coronary cusp.

At 1008, the method includes deploying the radially compressed prosthetic heart valve with the delivery apparatus to radially expand and implant the prosthetic heart valve in an annulus of the native valve such that the cusps of the prosthetic heart valve are circumferentially aligned with the cusps of the native valve. Consequently, the commissures of the prosthetic heart valve are also aligned with the commissures of the native valve.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve. A prosthetic valve may also be introduced via carotid, subclavian, and axiallary arteries.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A method comprising: advancing a distal end portion of a delivery apparatus toward a native valve of a heart, wherein a prosthetic heart valve is radially compressed around a valve mounting portion of the distal end portion of the delivery apparatus; visualizing under long axis fluoroscopy, within an imaging view, a position of one or more radiopaque markers relative to one or more cusps of the native valve, the one or more radiopaque markers disposed on the distal end portion of the delivery apparatus, and wherein at least one radiopaque marker of the one or more radiopaque markers corresponds to a location of a specified cusp of the prosthetic heart valve; at or proximate to the native valve, rotating the distal end portion of the delivery apparatus until the one or more radiopaque markers are circumferentially aligned with a middle of the one or more cusps of the native valve; and deploying the radially compressed prosthetic heart valve with the delivery apparatus to radially expand and implant the prosthetic heart valve in an annulus of the native valve such that the cusps of the prosthetic heart valve are circumferentially aligned with the cusps of the native valve.

Example 2. The method of any example herein, particularly example 1, further comprising, prior to the visualizing, obtaining a 3D image of the heart and selecting the imaging view based on the 3D image such that a specified cusp of the native valve is positioned in a known location in the selected imaging view, and wherein the rotating includes rotating the distal end portion of the delivery apparatus until one marker of the one or more radiopaque markers is aligned with a middle of the specified cusp of the native valve in the known location in the selected imaging view.

Example 3. The method of any example herein, particularly either example 1 or example 2, wherein the imaging view is a three-cusp imaging view where a right coronary cusp of the native valve disposed in a center of the imaging view, and wherein the rotating includes rotating the distal end portion of the delivery apparatus until one marker of the one or more radiopaque markers appears to be positioned anteriorly in the imaging view and is aligned with the middle of the right coronary cusp.

Example 4. The method of any example herein, particularly either example 1 or example 2, wherein the imaging view is a cusp overlap view where a non-coronary cusp of the native valve is disposed on a left side of the imaging view while a right coronary cusp and left coronary cusp of the native valve are superimposed with one another on a right side of the imaging view, and wherein the rotating includes rotating the distal end portion of the delivery apparatus until one marker of the one or more radiopaque markers appears on a far left side of the imaging view at a middle of the non-coronary cusp.

Example 5. The method of any example herein, particularly any one of examples 1-4, wherein the one or more radiopaque markers include three radiopaque markers that are spaced 120 degrees apart from each other around a circumference of the distal end portion of the delivery apparatus.

Example 6. The method of any example herein, particularly any one of examples 1-4, wherein the one or more radiopaque markers include a pair of radiopaque markers that are spaced 180 degrees apart from each other around a circumference of the distal end portion of the delivery apparatus.

Example 7. The method of any example herein, particularly example 6, wherein the pair of radiopaque markers are linear markers configured as lines having one or more of a different width, length, or longitudinal position on the delivery apparatus.

Example 8. The method of any example herein, particularly example 6, wherein the pair of radiopaque markers include first marker configured as a linear marker and a second marker configured as a V-shaped marker.

Example 9. The method of any example herein, particularly any one of examples 1-4, wherein the one or more radiopaque markers include a single asymmetric radiopaque marker disposed on the distal end portion of the delivery apparatus.

Example 10. The method of any example herein, particularly example 9, wherein the single asymmetric radiopaque marker has a C shape.

Example 11. The method of any example herein, particularly any one of examples 1 -10, wherein the one or more radiopaque markers are mounted on or embedded within a distal end portion of a shaft of a balloon catheter of the delivery apparatus, the balloon catheter extending distally from a handle portion of the delivery apparatus.

Example 12. The method of any example herein, particularly example 11, wherein the shaft of the balloon catheter is an outer shaft to which a proximal end portion of an inflatable balloon of the delivery apparatus is mounted.

Example 13. The method of any example herein, particularly example 12, wherein the balloon catheter further comprises an inner shaft including a distal end portion that extends distally from a distal end of the outer shaft, the inner shaft extending coaxially through the outer shaft and through the inflatable balloon.

Example 14. The method of any example herein, particularly example 11, wherein the shaft of the balloon catheter is an inner shaft that extends coaxially through and distally to an outer shaft of the balloon catheter.

Example 15. The method of any example herein, particularly any one of examples 11-14, wherein rotating the distal end portion of the delivery apparatus includes rotating the balloon catheter relative to the handle portion.

Example 16. The method of any example herein, particularly any one of examples 11-15, wherein deploying the radially compressed prosthetic heart valve includes inflating an inflatable balloon mounted on a distal end portion of the balloon catheter.

Example 17. A method comprising: advancing a distal end portion of a delivery apparatus toward a native valve of a heart, wherein a prosthetic heart valve is radially compressed around a valve mounting portion of the distal end portion of the delivery apparatus; visualizing under long axis fluoroscopy, with a cusp overlap imaging view, a position of a radiopaque marker relative to a non-coronary cusp of the native valve that is disposed on a left side of the imaging view while a right coronary cusp and left coronary cusp of the native valve are superimposed with one another on a right side of the imaging view, the radiopaque marker disposed on the distal end portion of the delivery apparatus and corresponding to a location of a specified cusp of the prosthetic heart valve; at or proximate to the native valve, rotating the distal end portion of the delivery apparatus until the radiopaque marker appears on a far left side of the imaging view at a middle of the non-coronary cusp; and deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

Example 18. The method of any example herein, particularly example 17, wherein the radiopaque marker is one of three radiopaque markers that are spaced 120 degrees apart from each other around a circumference of the distal end portion of the delivery apparatus, each of the three radiopaque markers corresponding to a location of a different cusp of three cusps of the prosthetic heart valve.

Example 19. The method of any example herein, particularly example 17, wherein the radiopaque marker is one of a pair of radiopaque markers that are spaced 180 degrees apart from each other around a circumference of the distal end portion of the delivery apparatus.

Example 20. The method of any example herein, particularly example 19, wherein the pair of radiopaque markers are linear markers configured as lines having a different width or length, and wherein the rotating includes rotating the distal end portion of the delivery apparatus until the pair of radiopaque markers are spaced 180 degrees apart from one another on opposite sides of the imaging view.

Example 21. The method of any example herein, particularly example 17, wherein the radiopaque marker is a single asymmetric marker disposed on the distal end portion of the delivery apparatus.

Example 22. The method of any example herein, particularly example 21, wherein the single asymmetric marker has a C shape, and wherein the rotating includes rotating the distal end portion of the delivery apparatus until the radiopaque marker appears as a line on the far left side of the imaging view at the middle of the non-coronary cusp.

Example 23. The method of any example herein, particularly any one of examples 17-22, wherein the radiopaque marker is mounted on or embedded within a distal end portion of a shaft of a balloon catheter of the delivery apparatus, the balloon catheter extending distally from a handle portion of the delivery apparatus.

Example 24. The method of any example herein, particularly example 23, wherein the shaft of the balloon catheter is an outer shaft to which a proximal end portion of an inflatable balloon of the delivery apparatus is mounted.

Example 25. The method of any example herein, particularly example 24, wherein the balloon catheter further comprises an inner shaft including a distal end portion that extends distally from a distal end of the outer shaft, the inner shaft extending coaxially through the outer shaft and through the inflatable balloon.

Example 26. The method of any example herein, particularly example 23, wherein the shaft of the balloon catheter is an inner shaft that extends coaxially through and distally to an outer shaft of the balloon catheter.

Example 27. The method of any example herein, particularly any one of examples 23-26, wherein rotating the distal end portion of the delivery apparatus includes rotating the balloon catheter relative to the handle portion.

Example 28. The method of any examples herein, particularly any one of examples 23-27, wherein deploying the radially compressed prosthetic heart valve includes inflating an inflatable balloon mounted on a distal end portion of the balloon catheter.

Example 29. A method comprising: obtaining a 3D image of a heart and based on the obtained 3D image, selecting a long axis fluoroscopic imaging view that positions a right coronary cusp of a native valve of the heart in a center of the imaging view, anterior to a left-non commissure of the native valve; advancing a distal end portion of a delivery apparatus toward the native valve of the heart, wherein a prosthetic heart valve is radially compressed around a valve mounting portion of the distal end portion of the delivery apparatus; visualizing under long axis fluoroscopy, within the selected imaging view, a position of a radiopaque marker relative to the right coronary cusp of the native valve, the radiopaque marker disposed on the distal end portion of the delivery apparatus and corresponding to a location of a specified cusp of the prosthetic heart valve; at or proximate to the native valve, rotating the distal end portion of the delivery apparatus until the radiopaque marker appears to be positioned anteriorly in the imaging view and is aligned with the middle of the right coronary cusp; and deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

Example 30. The method of any example herein, particularly example 29, wherein the radiopaque marker is asymmetric about a longitudinal axis of the marker.

Example 31. The method of any example herein, particularly example 30, wherein the radiopaque marker is shaped as a C.

Example 32. The method of any example herein, particularly example 30, wherein the radiopaque marker is shaped as a greater than or less than sign.

Example 33. The method of any example herein, particularly any one of examples 30-32, wherein the rotating includes rotating the delivery apparatus until the radiopaque marker is centered in the imaging view and is in a predetermined orientation of two possible readable orientations that indicates the radiopaque marker is positioned anteriorly.

Example 34. The method of any example herein, particularly any one of examples 29-33, wherein the radiopaque marker is one of a pair of radiopaque markers that are spaced 180 degrees apart from one another around a circumference of the distal end portion of the delivery apparatus.

Example 35. The method of any example herein, particularly any one of examples 29-33, wherein the radiopaque marker is one of three radiopaque markers that are spaced 120 degrees apart from one another around a circumference of the distal end portion of the delivery apparatus, and wherein each marker of the three radiopaque markers corresponds to a location of a different cusp of three cusps of the prosthetic heart valve.

Example 36. The method of any example herein, particularly example 35, wherein all three of the three radiopaque markers have a same shape.

Example 37. The method of any example herein, particularly example 35, wherein the three radiopaque markers have different shapes.

Example 38. The method of any example herein, wherein the method is performed on a living animal or on a non-living simulation.

Example 39. A method of treating a heart on a simulation, wherein the method includes the method of any example herein, particularly any one of examples 1-37.

Example 40. A method comprising sterilizing the prosthetic heart valve, apparatus, and/or assembly of any example.

The features described herein with regard to any example can be combined with other features described in any one or more of the other examples, unless otherwise stated. For example, any one or more of the features of one method can be combined with any one or more features of another method.

In view of the many possible examples to which the principles of the disclosed technology may be applied, it should be recognized that the illustrated examples are only preferred examples of the disclosed technology and should not be taken as limiting the scope of the claimed subject matter. Rather, the scope of the claimed subject matter is defined by the following claims and their equivalents.

We claim:

1. A method comprising:
advancing a distal end portion of a delivery apparatus toward a native valve of a heart, wherein a prosthetic heart valve is radially compressed around a valve mounting portion of the distal end portion of the delivery apparatus;
visualizing under long axis fluoroscopy, within an imaging view, a position of one or more radiopaque markers relative to one or more cusps of the native valve, the one or more radiopaque markers disposed on the distal end portion of the delivery apparatus, and wherein at least one radiopaque marker of the one or more radiopaque markers corresponds to a location of a specified cusp of the prosthetic heart valve;
at or proximate to the native valve, rotating the distal end portion of the delivery apparatus until the one or more radiopaque markers are circumferentially aligned with a middle of the one or more cusps of the native valve; and
deploying the radially compressed prosthetic heart valve with the delivery apparatus to radially expand and implant the prosthetic heart valve in an annulus of the native valve such that the cusps of the prosthetic heart valve are circumferentially aligned with the cusps of the native valve.

2. The method of claim 1, further comprising, prior to the visualizing, obtaining a 3D image of the heart and selecting the imaging view based on the 3D image such that a specified cusp of the native valve is positioned in a known location in the selected imaging view, and wherein the rotating includes rotating the distal end portion of the delivery apparatus until one marker of the one or more radiopaque markers is aligned with a middle of the specified cusp of the native valve in the known location in the selected imaging view.

3. The method of claim 1, wherein the imaging view is a three-cusp imaging view where a right coronary cusp of the native valve is disposed in a center of the imaging view, and wherein the rotating includes rotating the distal end portion of the delivery apparatus until one marker of the one or more radiopaque markers appears to be positioned anteriorly in the imaging view and is aligned with the middle of the right coronary cusp.

4. The method of claim 1, wherein the imaging view is a cusp overlap view where a non-coronary cusp of the native valve is disposed on a left side of the imaging view while a right coronary cusp and left coronary cusp of the native valve are superimposed with one another on a right side of the imaging view, and wherein the rotating includes rotating the distal end portion of the delivery apparatus until one marker of the one or more radiopaque markers appears on a far left side of the imaging view at a middle of the non-coronary cusp.

5. The method of claim 1, wherein the one or more radiopaque markers include three radiopaque markers that are spaced 120 degrees apart from each other around a circumference of the distal end portion of the delivery apparatus.

6. The method of claim 1, wherein the one or more radiopaque markers include a pair of radiopaque markers that are spaced 180 degrees apart from each other around a circumference of the distal end portion of the delivery apparatus.

7. The method of claim 6, wherein the pair of radiopaque markers are linear markers configured as lines having one or more of a different width, length, or longitudinal position on the delivery apparatus.

8. The method of claim 6, wherein the pair of radiopaque markers include first marker configured as a linear marker and a second marker configured as a V-shaped marker.

9. The method of claim 1, wherein the one or more radiopaque markers include a single asymmetric radiopaque marker disposed on the distal end portion of the delivery apparatus.

10. The method of claim 1, wherein the one or more radiopaque markers are mounted on or embedded within a distal end portion of a shaft of a balloon catheter of the delivery apparatus, the balloon catheter extending distally from a handle portion of the delivery apparatus.

11. The method of claim 10, wherein the shaft of the balloon catheter is an outer shaft to which a proximal end portion of an inflatable balloon of the delivery apparatus is mounted.

12. The method of claim 10, wherein the shaft of the balloon catheter is an inner shaft that extends coaxially through and distally to an outer shaft of the balloon catheter.

13. A method comprising:
advancing a distal end portion of a delivery apparatus toward a native valve of a heart, wherein a prosthetic heart valve is radially compressed around a valve mounting portion of the distal end portion of the delivery apparatus;
visualizing under long axis fluoroscopy, with a cusp overlap imaging view, a position of a radiopaque marker relative to a non-coronary cusp of the native valve that is disposed on a left side of the imaging view while a right coronary cusp and left coronary cusp of the native valve are superimposed with one another on a right side of the imaging view, the radiopaque marker disposed on the distal end portion of the delivery apparatus and corresponding to a location of a specified cusp of the prosthetic heart valve;
at or proximate to the native valve, rotating the distal end portion of the delivery apparatus until the radiopaque marker appears on a far left side of the imaging view at a middle of the non-coronary cusp; and
deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

14. The method of claim 13, wherein the radiopaque marker is one of three radiopaque markers that are spaced 120 degrees apart from each other around a circumference of the distal end portion of the delivery apparatus, each of the three radiopaque markers corresponding to a location of a different cusp of three cusps of the prosthetic heart valve.

15. The method of claim 13, wherein the radiopaque marker is one of a pair of radiopaque markers that are spaced 180 degrees apart from each other around a circumference of the distal end portion of the delivery apparatus.

16. The method of claim 15, wherein the pair of radiopaque markers are linear markers configured as lines having a different width or length, and wherein the rotating includes rotating the distal end portion of the delivery apparatus until the pair of radiopaque markers are spaced 180 degrees apart from one another on opposite sides of the imaging view.

17. The method of claim 13, wherein the radiopaque marker is a single asymmetric marker disposed on the distal end portion of the delivery apparatus.

18. The method of claim 17, wherein the single asymmetric marker has a C shape, and wherein the rotating includes rotating the distal end portion of the delivery apparatus until the radiopaque marker appears as a line on the far-left side of the imaging view at the middle of the non-coronary cusp.

19. A method comprising:
obtaining a 3D image of a heart and based on the obtained 3D image, selecting a long axis fluoroscopic imaging view that positions a right coronary cusp of a native valve of the heart in a center of the imaging view, anterior to a left-non commissure of the native valve;
advancing a distal end portion of a delivery apparatus toward the native valve of the heart, wherein a prosthetic heart valve is radially compressed around a valve mounting portion of the distal end portion of the delivery apparatus;
visualizing under long axis fluoroscopy, within the selected imaging view, a position of a radiopaque marker relative to the right coronary cusp of the native valve, the radiopaque marker disposed on the distal end portion of the delivery apparatus and corresponding to a location of a specified cusp of the prosthetic heart valve;
at or proximate to the native valve, rotating the distal end portion of the delivery apparatus until the radiopaque marker appears to be positioned anteriorly in the imaging view and is aligned with the middle of the right coronary cusp; and
deploying the radially compressed prosthetic heart valve with the delivery apparatus to radially expand and implant the prosthetic heart valve in an annulus of the native valve such that cusps of the prosthetic heart valve are circumferentially aligned with cusps of the native valve.

20. The method of claim 19, wherein the radiopaque marker is asymmetric about a longitudinal axis of the marker, and wherein the radiopaque marker is shaped as a C, a greater than sign, or a less than sign.
